(19) United States
(12) Patent Application Publication
(10) Pub. No.: US 2024/0277610 A1
(43) Pub. Date: Aug. 22, 2024

(54) BIOMATERIALS COATING FOR ON-DEMAND BACTERIA DELIVERY AND METHOD TO TREAT COLITIS AND IRRITABLE BOWEL SYNDROME

(71) Applicant: Wisconsin Alumni Research Foundation, Madison, WI (US)

(72) Inventors: Quanyin Hu, Madison, WI (US); Jun Liu, Madison, WI (US)

(73) Assignee: Wisconsin Alumni Research Foundation, Madison, WI (US)

(21) Appl. No.: 18/570,690

(22) PCT Filed: Jun. 8, 2022
(86) PCT No.: PCT/US2022/032640

§ 371 Date: Dec. 15, 2023

Related U.S. Application Data
(60) Provisional application No. 63/210,533, filed on Jun. 15, 2021.

(51) Int. Cl. A61K 9/00; A61K 9/48; A61K 35/00; A61K 35/741

(52) U.S. Cl. CPC A61K 9/0065; A61K 9/4816; A61K 35/741; A61K 2035/115

(57) ABSTRACT

A method of encapsulating a probiotic including the steps of (a) coating a probiotic with a compound or composition of matter dimensioned and configured to increase adhesion of the probiotic to intestinal mucosa; and (b) coating the probiotic of step (a) with a polymer or copolymer that resists degradation in the stomach and at least partially degrades in the small intestine: the composition of matter resulting therefrom, methods of using the composition of matter to deliver probiotics to the small intestine by mouth, and methods of treating colitis and IBD by administering the compositions of matter by mouth to a subject.

### CROSS-REFERENCE TO RELATED APPLICATIONS

Priority is hereby claimed to U.S. provisional patent application Ser. No. 63/210,533, filed Jun. 15, 2021, which is incorporated herein by reference.

### BACKGROUND

Bacteria, as one of the most abundant organisms in the human body, dynamically participate in various physiological activities (1-3). Recently, there is increasing evidence showing that the dysbiosis of the commensal bacteria in the GI tract can significantly impact human health by inducing various disorders, including inflammatory bowel disease (IBD), obesity, and autoimmune diseases (4-6). The complexity of the intestinal flora ecosystem imposes challenges on regulating the homeostasis of microbiota in the GI tract (7-9). Furthermore, the microbiome in the GI tract can be easily perturbed by food and medication intake, which potentially exacerbates the severity of the diseases in the patient where necessary medication is inevitable (10-12). Thus, the regulation of gut microbiota under the complex interplay of multiple influencing factors is the key for effective treatment of diseases related to the imbalance of intestinal flora.

As beneficial bacteria strains, probiotics have shown tremendous treatment efficacy against GI tract-related diseases, such as irritable bowel disease (“IBD”), in preclinical and clinical studies (13, 14). Probiotics can mitigate the severity and maintain the remission of IBD through a variety of mechanisms, such as regulating intestinal flora homeostasis, inhibiting colonization of pathogenic bacteria, and altering GI immunity by decreasing inflammatory cytokines generation (15, 16). In clinic, bacteriotherapy has achieved promising treatment outcomes in patients with recurrent Clostridium difficile colitis after transplanting fecal microbiota from a healthy donor (17). Despite the growing interest and increasing clinical trials in leveraging live bacteria, including probiotics, to treat GI tract disorders, its widespread application is dampened by its limited success and elusive treatment mechanism, partially attributed to the probiotics' low availability and limited intestinal colonization after oral administration (18-20). Specifically, the low gastric pH and harsh intestinal environment have significantly reduced the viability and proliferation of probiotics (21-23). Furthermore, the low retention time of delivered probiotics due to the rapid transit time in the gastro-intestinal (“GI”) tract contributes to the unsatisfactory treatment efficacy, resulting in insufficient regulation of intestinal flora (24, 25). Thus, the protection of probiotics after oral administration and enhanced retention in the GI tract is critical to improve the effectiveness of treatment. This remains a long-felt and unmet need.

### SUMMARY

Bacteria-based therapy has shown great promise in treating various diseases by regulating intestinal flora balance in pre-clinical and clinical studies. However, weak resistance against stress in the GI tract and limited retention time in the intestine result in low bacterial availability and persistence, restricting further clinical application. Disclosed herein is a double-layer coating strategy employing tannic acid (“TA”) and at least one copolymer comprising two or more of methyl acrylate, methyl methacrylate, and/or methacrylic acid encapsulating probiotics to address the delivery challenges after oral administration. An exemplary such copolymer is “EUDRAGIT”®-brand L100 copolymer (see below for further description). For sake of brevity only, any such copolymers (i.e., copolymers comprising two or more of methyl acrylate, methyl methacrylate, and/or methacrylic acid) shall be referred to herein as “L100.” The Escherichia coli Nissle 1917 (“EcN”) encapsulated by a TA layer and a L100 copolymer layer display robust resistance against the harsh environment of the GI tract. Furthermore, the pH-responsive degradation of the outer copolymer layer leads to the selective delivery of TA-EcN to the intestine, where the strong mucoadhesive capability of the TA layer prolongs the retention time of EcN without compromising the viability and proliferation capabilities of EcN, resulting in superior efficacy of prophylactic and treatment against colitis. To mitigate the potential side effects caused by long-term mucoadhesion of TA, the TA layer can be further removed by addition of ethylenediaminetetraacetic acid (“EDTA”). The spatiotemporal delivery of probiotics through double-layer encapsulation can significantly augment the therapeutic efficacy of bacteria-based therapy against GI tract-related diseases.

Thus, disclosed herein is a method of encapsulating a probiotic, the method comprising:
(a) overlaying a probiotic with a first layer comprising a compound or composition of matter dimensioned and configured to increase adhesion of the probiotic to intestinal mucosa; and
(b) overlaying the probiotic of step (a) with a second layer comprising a compound or composition that resists degradation in the stomach and at least partially degrades in the small intestine.

Step (a) of the method comprises overlaying the probiotic with a tannin or tannic acid. Step (b) comprises overlaying the probiotic of step (a) with a copolymer comprising monomers selected from the group consisting of methyl acrylate, methyl methacrylate, and methacrylic acid. In some embodiments, step (b) comprises overlaying the probiotic of step (a) with a EUDRAGIT®-brand copolymer, such as a EUDRAGIT®-L100-brand copolymer.

The probiotic of the method comprises E. coli cells, such as Escherichia coli Nissle 1917 cells.

Also disclosed herein is a composition of matter made by any of the methods described herein. The composition of matter comprising:
a probiotic overlayed with a first layer comprising a compound or composition of matter dimensioned and configured to increase adhesion of the probiotic to intestinal mucosa; and
a second layer overlaying the first layer and comprising a polymer or copolymer that resists degradation in the stomach and at least partially degrades in the small intestine.

In the composition of matter, the first layer comprises a tannin or tannic acid. The second layer comprises a copolymer comprising monomers selected from the group consisting of methyl acrylate, methyl methacrylate, and methacrylic acid. In some embodiments, the second layer comprises a EUDRAGIT®-brand copolymer, such as a EUDRAGIT®-L100-brand copolymer.

The probiotic of the composition of matter comprises E. coli cells, such as Escherichia coli Nissle 1917 cells.

Also disclosed herein is a method of delivering a probiotic to the intestine, the method comprising administering by mouth any of the composition of matter described herein.

Also disclosed herein is a method of treating irritable bowel syndrome and colitis, the method comprising administering to a mammal, by mouth, any of the composition of matter described herein, wherein the composition of matter comprises a probiotic in an amount effective to inhibit irritable bowel syndrome and colitis.

### BRIEF DESCRIPTION OF THE DRAWINGS

FIGS. 1A-1G: Schematic illustration and characterization of L100-TA-EcN. FIG. 1A: Schematic of the protection capability of L100-TA-EcN, selective release of TA-EcN in the intestine for mucoadhesion, and removal of TA layer with the addition of EDTA. (i) The L100 layer protects the bacteria from insults in the acidic gastric environment and degrades after being extruded to the intestine due to pH-triggered degradation. (ii) The exposed TA-EcN adheres on mucosal epithelial layers for prolonging the retention time of bacteria based on the interaction between TA and mucin. (iii) TA layer can be removed on-demand with the addition of EDTA. Sizes (FIG. 1B) and Zeta potentials (FIG. 1C) of EcN, TA-EcN, and L100-TA-EcN measured by DLS. Data are presented as the mean±SD (n=3). FIG. 1D: TEM images of EcN, TA-EcN and L100-TA-EcN. Scale bar, 1 μm. FIG. 1E: LSCM images of EcN, TA-EcN and L100-TA-EcN. The EcN cells were labeled with Hoechst 33342. The TA layer was stained with BSA-Alexa Fluor 647. The L100 layer was labeled with FITC. Scale bar, 10 μm. FIG. 1F: The growth curve of EcN and TA-EcN in LB medium at 37° C., and the OD₆₀₀ was recorded in 30 min intervals by a microplate reader. FIG. 1G: The growth curve of EcN and L100-TA-EcN in LB medium at 37° C. monitored by OD₆₀₀ in 30 min intervals.

FIGS. 2A-2F. Evaluation of L100-TA-EcN against environmental stress. FIGS. 2A-2D: Survival of EcN and L100-TA-EcN after exposure to the following solutions: FIG. 2A: SGF (pH 1.6) supplemented with pepsin (0.32%). FIG. 2B: SIF (pH 6.8) supplemented with trypsin (10 mg/mL). FIG. 2C: a strong basic LB medium (pH 10.6). and FIG. 2D: LB medium with 0.05 mg/mL of Ampicillin. The number of surviving bacteria was quantified by spreading 50 μL of serially diluted samples onto LB agar plates and incubating at 37° C. for 24 h. FIG. 2E: The growth curves of EcN and L100-TA-EcN cultured in LB medium with H₂O₂ (80 mM). FIG. 2F: The growth curves of EcN and L100-TA-EcN cultured in LB medium with 0.5% bile salt. For all growth curves recording, the OD₆₀₀ value was monitored in 30 min intervals by a microplate reader. Data are presented as the mean±SD (n=3). Statistical analysis was performed using Student's t-test. *P<0.05, **P<0.01, ***P<0.001.

FIGS. 3A-3H. Mucoadhesive capability of L100-TA-EcN. FIG. 3A: Fluorescence images of ex vivo intestines after incubation with rhodamine B-labeled EcN, TA-EcN, and TA-EcN with the addition of EDTA. FIG. 3B: Region-of-Interest analysis of fluorescence intensities of the intestines. FIG. 3C: Bioluminescence images of the ex vivo mice GI tracts at 4 h post-administration of various luciferase-expressed bacteria formulations. FIG. 3D: Region-of-Interest analysis of bioluminescence intensities of the intestines. FIG. 3E: Bioluminescence images of mice administered with various luciferase-expressed bacteria formulations at different time points. In the EDTA group, mice were administered EDTA solution via oral gavage at 4 h and 12 h, respectively. FIG. 3F: Bioluminescence decay curve of bioluminescence intensities of the mice at all pre-determined timepoints through Region-of-Interest analysis. FIG. 3G: Bioluminescence images of mice GI tracts at 120 h post-administration of various luciferase-expressed bacteria formulations. FIG. 3H: Region-of-Interest analysis of bioluminescence intensities of the mice GI tract at 120 h. Data are represented as mean±SD (n=3). Statistical analysis was performed using one-way ANOVA. *P<0.05, **P<0.01, ***P<0.001.

FIGS. 4A-4E. Therapeutic efficacy against DSS-induced mouse colitis model. FIG. 4A: Schematic of the treatment plan. EcN formulations were administered by oral gavage, and 3% DSS was administered via drinking water. FIG. 4B: Percentage weight changes after administration of various bacteria formulations. The mice without DSS administration served as the control. FIG. 4C: Colon length of the colitis-bearing mice after treatment with various bacteria formulations on day 4. FIG. 4D: Histopathology score of the colons of the colitis-bearing mice after treatment with various bacteria formulations. FIG. 4E: Representative histology images of colon tissues stained with H&E. Scale bar: 100 μm. Data are presented as mean±SD (n=6). Statistical analysis was performed using one-way ANOVA. *P<0.05, **P<0.01, ***P<0.001.

FIGS. 5A-5E. Preventative efficacy against DSS-induced mouse colitis model. FIG. 5A: Schematic of administration schedule. EcN formulations were administered by oral gavage daily, and 3% DSS was administered via drinking water from day 0 to day 7. FIG. 5B: Percentage weight changes of the colitis-bearing mice treated with various EcN formulations. The mice without DSS administration served as control. FIG. 5C: Colon length of the colitis-bearing mice after treating with various bacteria formulations on day 10. FIG. 5D: Histopathology score of the colons of the colitis-bearing mice after treating with various bacteria formulations. FIG. 5E: Representative histology images of colon tissues stained with H&E. Scale bar: 100 μm. Data are presented as mean±SD (n=6). Statistical analysis was performed using one-way ANOVA. *P<0.05, **P<0.01, ***P<0.001.

FIG. 6. The chemical structure of tannic acid.

FIG. 7. Size distributions of EcN, TA-EcN, and L100-TA-EcN measured by dynamic light scattering (“DLS”).

FIG. 8. The confocal microscopy images of TA-EcN. The bacteria were labeled with Hoechst 33342, and the TA layer was labeled with Alexa fluor 647. Scale bar, 10 μm.

FIG. 9. Bacteria viability of EcN after encapsulation of the TA layer measured by CCK-8 assay. Non-coated EcN served as the control. The OD₄₅₀ was recorded in 1 h intervals by a microplate reader. Data are presented as mean±SD (n=3). Statistical analysis was performed using Student's t-test. n.s. P>0.05.

FIG. 10. The confocal images of L100-TA-EcN. The bacteria were labeled with Hoechst 33342, and the L100 layer was labeled with FITC. Scale bar, 10 μm.

FIG. 11. The growth curve of EcN incubated with different concentrations of L100 in the LB medium. The EcN with addition of L100 served as control. (n=3).

FIG. 12. Bacteria viability of EcN after encapsulation with both TA and L100 layers measured by CCK-8 assay. Non-coated EcN served as the control. Data are presented as mean±SD (n=3). Statistical analysis was performed using Student's t-test. n.s. P>0.05. ***P<0.001.

FIGS. 13A-13C. The controllable degradation of L100 and TA layers. FIG. 13A: Schematic illustration for preparation and degradation of L100 and TA layers. FIG. 13B: TEM images of EcN, EcN following encapsulation of the L100 and TA layers, L100-TA-EcN after incubation in PBS for 4 h, and further incubation in EDTA for 4 h. Scale bar: 1 μm. FIG. 13C: LSCM images of L100-TA-EcN following incubation in PBS for 4 h, staining with BSA-Alexa 647, and further incubation in EDTA solution for 4 h. The EcN cells were labeled with Hoechst 33342. The L100 layer was labeled with FITC. The TA layer was labeled with Alexa fluor 647. Scale bar: 10 μm.

FIG. 14. Reactive oxygen species (“ROS”) levels in EcN and L100-TA-EcN measured by DCFH-DA assay after induction with H₂O₂ (1 mM) for 1 h. All the ROS levels were normalized to EcN without H₂O₂ induction. Data are presented as mean±SD (n=3). Statistical analysis was performed using Student's t-test. ***P<0.001.

FIG. 15. Changes in mice body weight after administration of various bacteria formulations. (n=3).

FIG. 16. The mice intestinal length after administration of various bacteria formulations. (n=3).

FIG. 17. Histological images of heart, liver, spleen, lung and kidney tissues stained with H&E in various mice groups treated with different bacteria formulations. Scale bar, 100 μm.

### DETAILED DESCRIPTION

Probiotic therapeutics exert beneficial effects on the host through modulating the homeostasis of the gut microbiota. However, low tolerance against the harsh environment of the GI tract and poor intestinal colonization encountered during oral delivery limited its clinical application. This highlights the need for advanced probiotics delivery systems. Disclosed herein is a double-layer coating strategy to protect probiotics from the complexity of the GI tract and to selectively release probiotics in the intestine. This is accomplished via a pH-responsive, time-delayed degradation of an outer shell or layer comprised of a copolymer as described herein and an inner shell or layer comprised of at least one tannin. This permits at least a portion of the encapsulated probiotic to pass through the harsh environment of the stomach and to be released (at least partially) in the small or large intestine. Furthermore, the tannin coating enhances mucoadhesion for prolonged retention of probiotics, leading to improved prophylactic and treatment efficacy against the DSS-induced mouse colitis model. (See below.) In addition, the adhesive tannin layer can be disassociated on-demand by the simple addition of EDTA. This double-layer coating approach is a platform technology that can be adapted to other living cells for spatiotemporal delivery to augment treatment efficacy.

### Abbreviations and Definitions

ANOVA=analysis of variance. BSA=bovine serum albumin. CCK-8=cell-counting kit 8 (a commercial cell-counting kit, available from APExBIO Technology, LLC, Houston, Texas, USA). DCFH-DA=dichlorodihydrofluorescein diacetate staining. DI=deionized. DLS=dynamic light scattering. DSS=dextran sulfate sodium. EcN cells=Escherichia coli Nissle 1917, EDTA=ethylenediaminetetraacetic acid. FITC=fluorescein isothiocyanate. GI=gastro-intestinal. IBD=irritable bowel disease. IPTG=isopropyl β-D-1-thiogalactopyranoside. L100=A copolymer comprising at least two monomers selected from the group consisting of methyl acrylate, methyl methacrylate, and methacrylic acid. An example of an L100-type copolymer is “EUDRAGIT”®-brand polymers (Evonik Industries, Parisppany, New Jersey, USA; “EUDRAGIT” is a registered trademark of Evonik Operations GmbH). The various “EUDRAGIT” branded products have different acidic or alkaline end groups, which allow for a pH-dependent dissolution by salt formation. LB medium=Luria-Bertani medium (also known as lysogeny broth). L100=a generic term broadly encompassing any copolymer comprising two or more of methyl acrylate, methyl methacrylate, and/or methacrylic acid. LSCM=laser scanning confocal microscopy. PBS=phosphate-buffered saline. ROS=reactive oxygen species. SD=standard deviation. SGF=simulated gastric fluid. SIF=simulated intestinal fluid (SIF). TA=tannic acid. TEM=transmission electron microscopy.

The word “tannin” is used broadly herein to denote a class of astringent, polyphenolic biomolecules comprising monomers selected from the group consisting of gallic acid (3,4,5-trihydroxybenzoic acid), phloroglucinol (benzene-1,3,5-triol), flavan-3-ols (sometimes referred to as flavanols, i.e., monomers having 2-phenyl-3,4-dihydro-2H-chromen-3-ol skeleton), and combinations thereof. Tannins typically have a molecular mass of from about 500 Da to about 20,000 Da for the largest proanthocyanidins (which are included in the definition). Tannic acid itself is the canonical example. Tannic acid has two naturally occurring isomers, quercitannic acid and gallotannic acid, which are included in the definition. Also included in the definition are hydrolyzable tannins, phlorotannins, condensed tannins, phlobatannins (C-ring isomerized condensed tannins), and oligo- and polystilbenes/stilbenoids.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite articles “a” and “an” mean “one or more.”

All combinations of method or process steps as used herein can be performed in any order, unless otherwise specified or clearly implied to the contrary by the context in which the referenced combination is made.

The methods disclosed herein can comprise, consist of, or consist essentially of the essential elements and limitations of the method disclosed, as well as any additional or optional ingredients, components, or limitations described herein or otherwise useful in the art of pharmaceutical formulations.

### Introduction

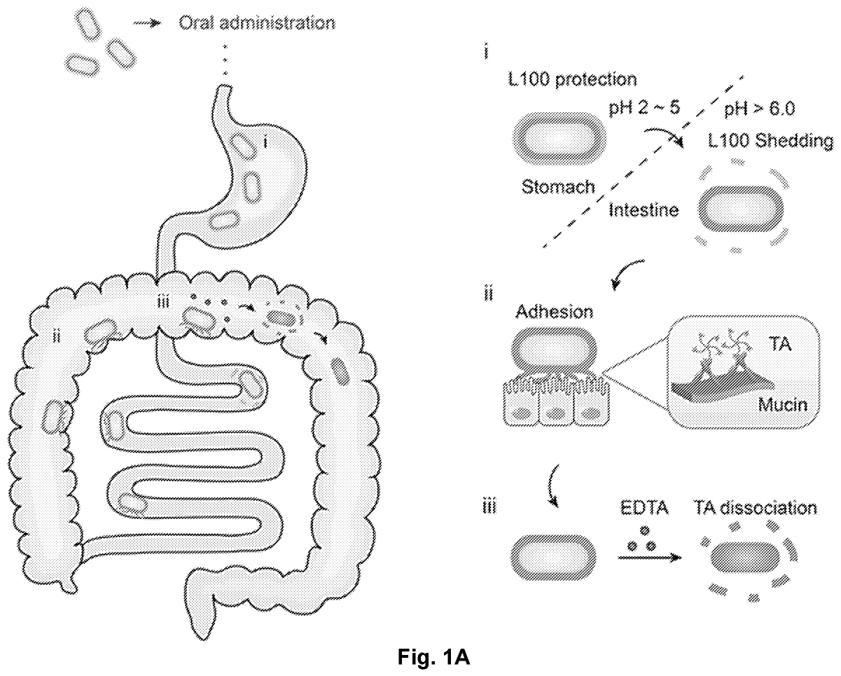

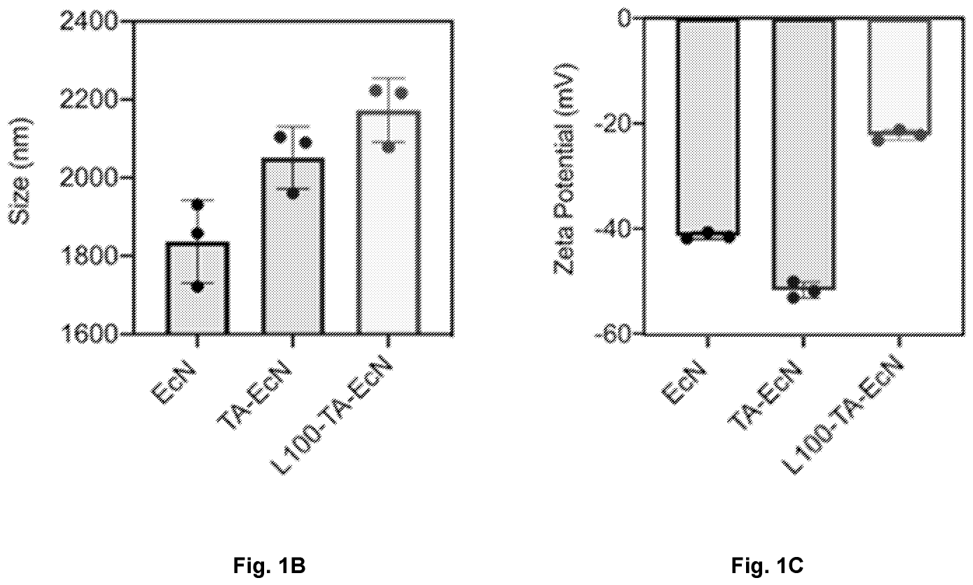

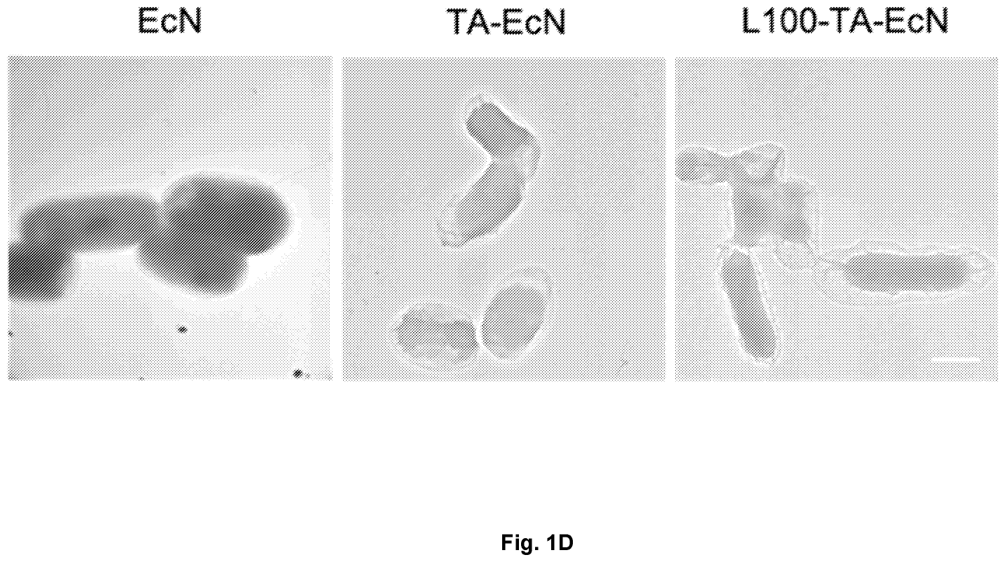

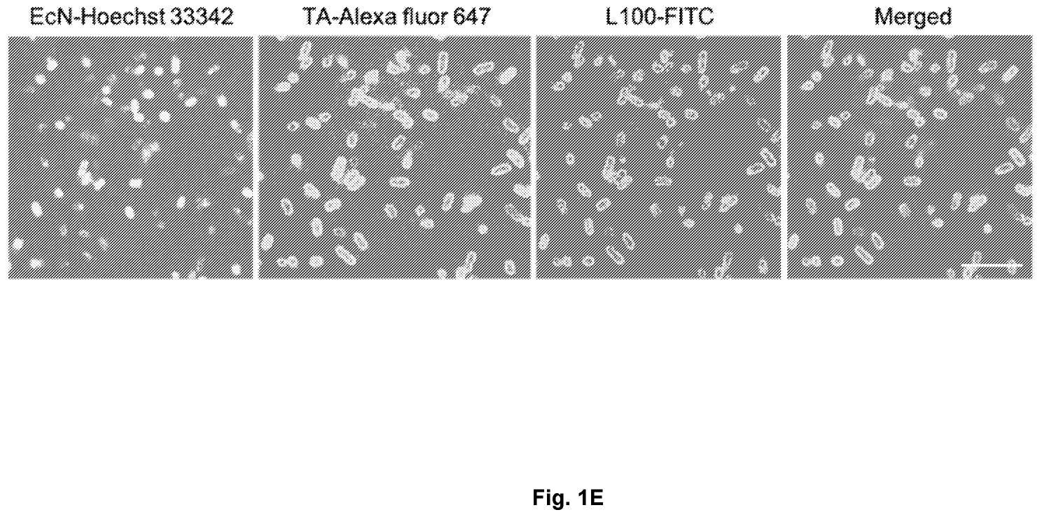

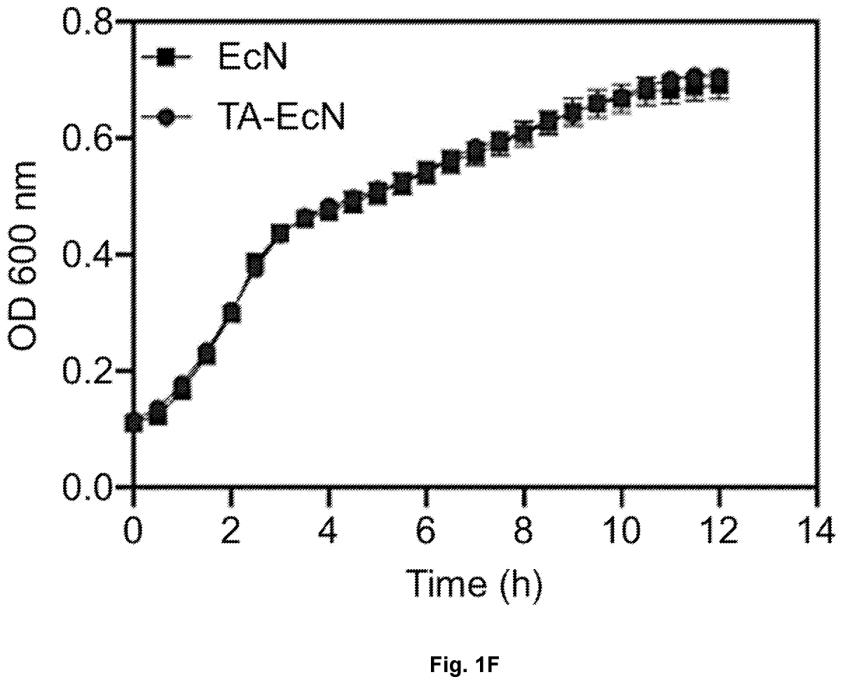

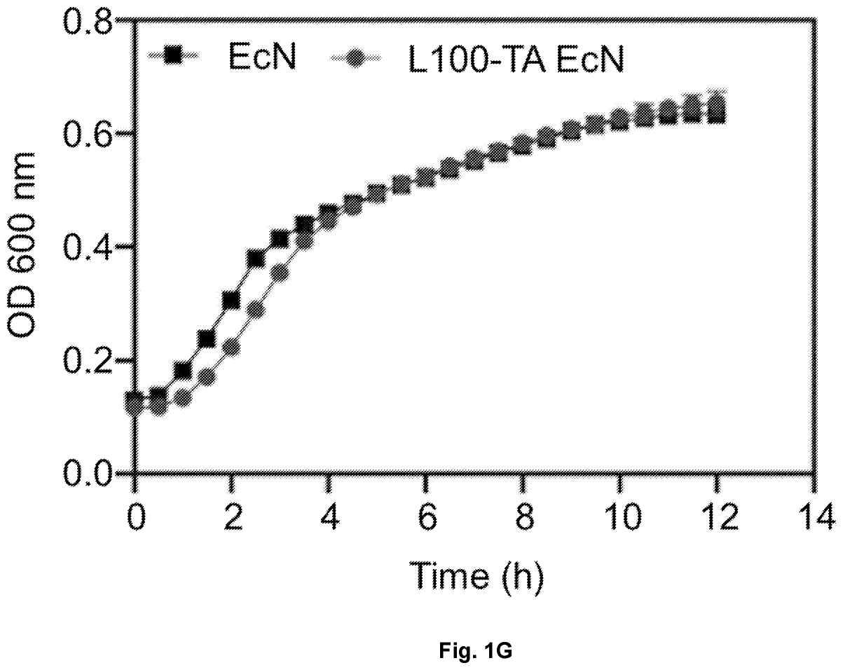

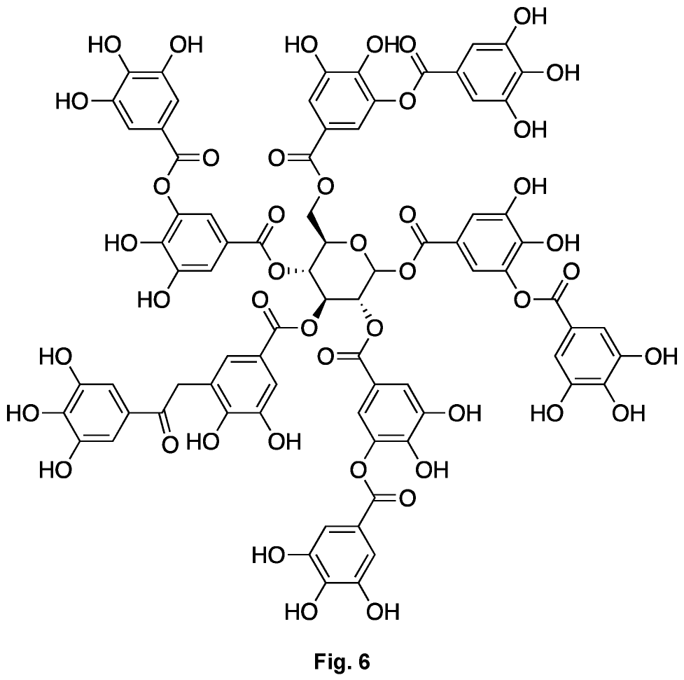

Disclosed herein is a probiotics coating method comprising encapsulating a probiotic (for example, EcN) with a protective layer to resist the harsh external environment of the GI tract and an adhesive layer to prolong retention time after accumulating in the GI tract (FIG. 1A). The protective layer is comprised of a copolymer comprising two or more of methyl acrylate, methyl methacrylate, and methacrylic acid. Eudragit®-brand L100, an enteric copolymer approved for use in pharmaceutical products by the U.S. Food and Drug Administration (26) was used as an exemplary copolymer to coat a probiotic. For brevity only, the disclosure will refer to EcN as an exemplary probiotic. L100-type copolymers will not dissolve in an acidic environment, thus protecting probiotics from gastric digestion. It disassociates and releases the probiotics once being extruded to the intestine (pH>6.0), leading to enhanced EcN availability in the GI tract. To increase the retention time of EcN, the EcN probiotic was coated with a polyphenol tannin layer. The tannin used in the example was tannic acid (TA) itself. See FIG. 6. TA is formed by crosslinking catechol groups on the TA with ferric ions. TA has been reported to possess strong adhesive properties contributed by the catechol groups that can form hydrogen bonds, covalent bonds, and/or π-π interactions with diverse substrates (27, 28). Thus, coating EcN probiotic with TA increases retention time of EcN in the GI tract by adhering the probiotic to the intestinal mucosa, without influencing the probiotics' growth and proliferation.

In addition to the intestinally degraded L100-type copolymer layer, EDTA was used to competitively chelate ferric ions to disassociate the tannin/TA layer. When taken together, the coating strategy creates a favorable milieu where probiotic cells are protected and selectively released in the GI tract for colonization resistance in when treating intestinal disorders such as IBD, colitis, and the like.

### The Method:

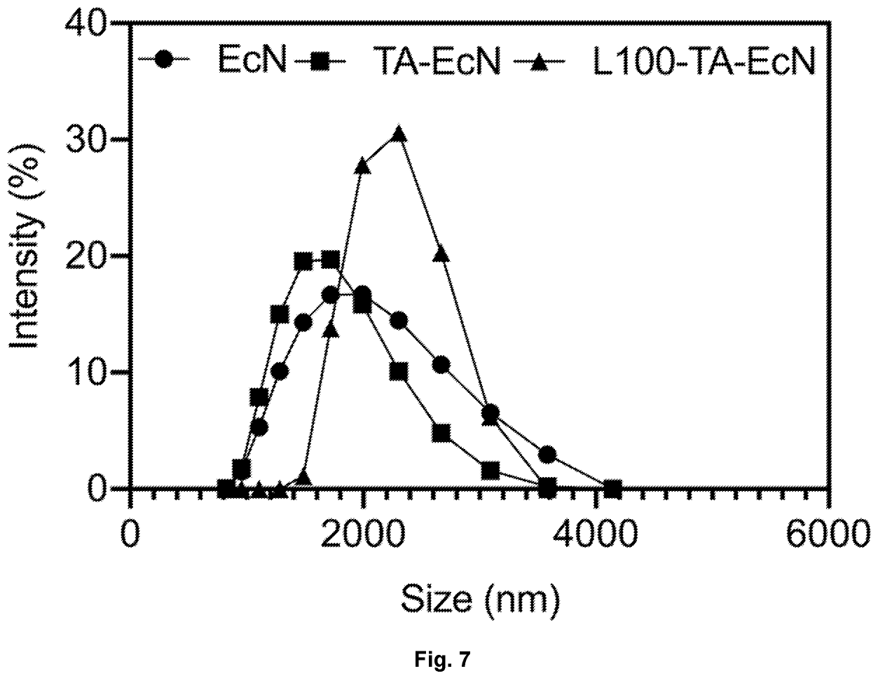

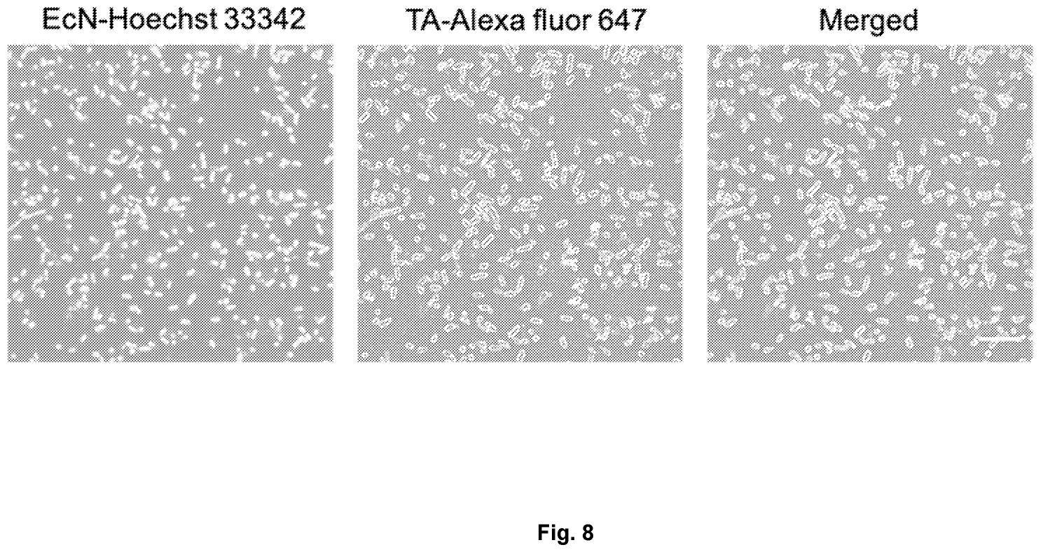

EcN, a well-known probiotic strain for regulating intestinal flora, was chosen as the model bacterial probiotic (29, 30). To coat the tannn layer onto the EcN probiotic, TA and ferric trichloride were sequentially mixed with EcN. The TA was crosslinked to form a compact layer on the surface of EcN with the assistance of ferric ions. The EcN was first characterized by dynamic light scanning (DLS) to measure changes in the size and surface charge after TA layer coating. The size of EcN (FIGS. 1B and 7) increased after decoration with the TA layer. The zeta potential (FIG. 1C) dropped, demonstrating the existence of the TA layer on the surface of EcN. A transparent outer shell was observed on the EcN under transmission electron microscopy (TEM), indicating that the TA layer was formed and coated on the surface of EcN (FIG. 1D). The decoration of the TA layer on the EcN was further characterized by laser scanning confocal microscopy (LSCM), as evidenced by the BSA-Alexa fluor 647 stained TA layer wrapped on the surface of EcN (FIGS. 1E and 8).

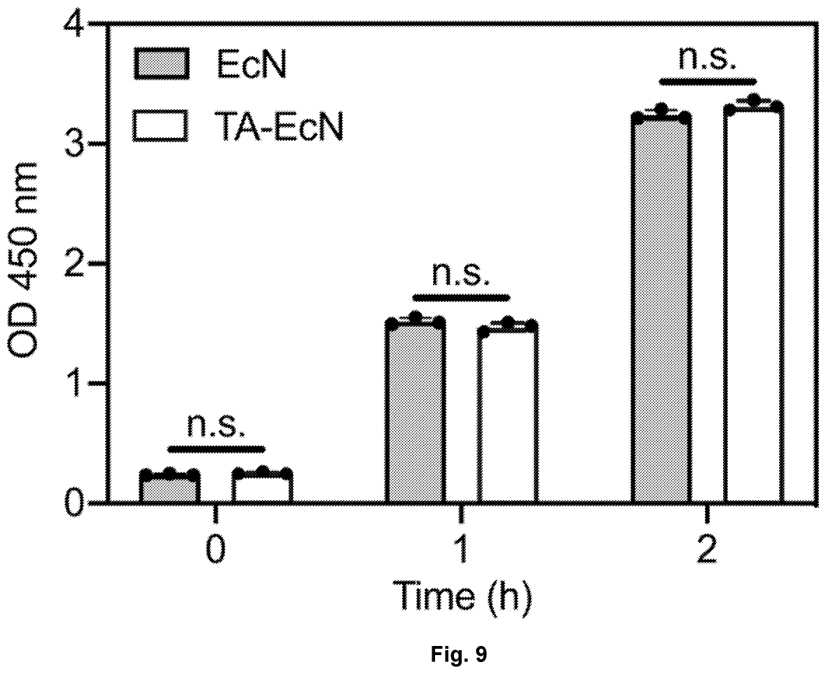

To examine whether TA coating affects the growth and viability of EcN cells, TA-EcN was incubated in a lysogeny broth (LB) medium, and the growth curve was monitored for 12 h via OD₆₀₀ values. As shown in FIG. 1F, a similar growth rate was found between TA-EcN and EcN, demonstrating that the TA layer had negligible influence on the growth and proliferation of EcN. A commercial cell counting kit (CCK-8) was utilized to evaluate the viability of TA-EcN. No significant difference was found between the viability of TA-EcN and untreated EcN, demonstrating the biocompatibility of the TA layer for bacteria coating. See FIG. 9.

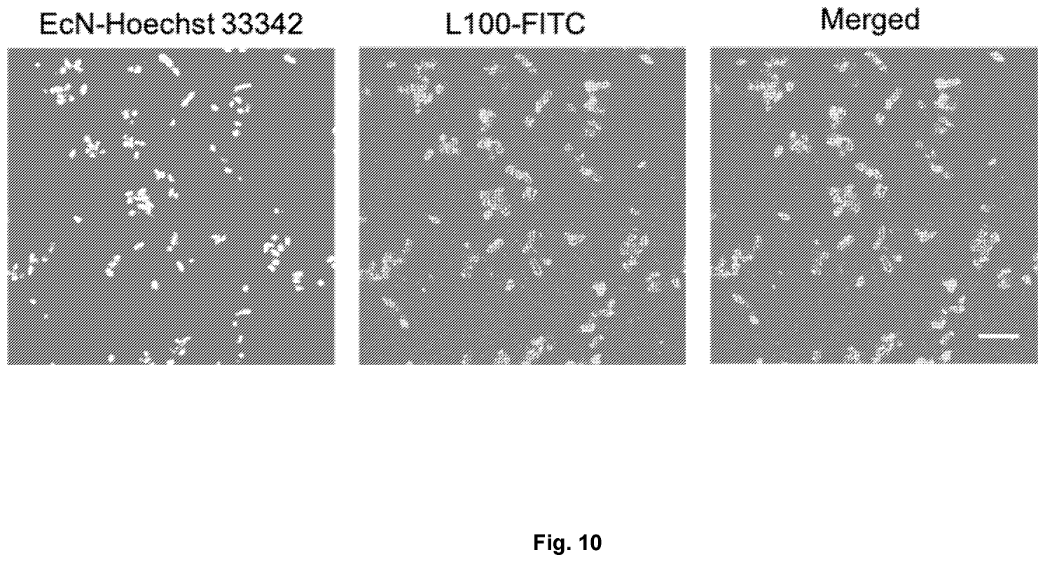

To encapsulate TA-EcN with an L100-type copolymer, the surface charge of TA-EcN was adjusted with positive calcium ions to facilitate the surface assembly of L100 copolymer through electrostatic interaction at pH 5.5. After L100 copolymer encapsulation, the size was increased from 2051 to 2171 nm (FIG. 1B), and zeta potential was increased from −51.7 to −22.2 mV measured by DLS (FIG. 1C). As shown in FIG. 1D, a thicker outer layer on L100-TA-EcN was observed under TEM when compared to TA-EcN, indicating the addition of the L100 layer on TA-EcN. The existence of the L100 copolymer outer shell was further confirmed by fluorescein isothiocyanate (FITC)-labeled L100 on the surface of Alexa fluor 647 labeled-TA-EcN via LSCM (FIGS. 1E and 10).

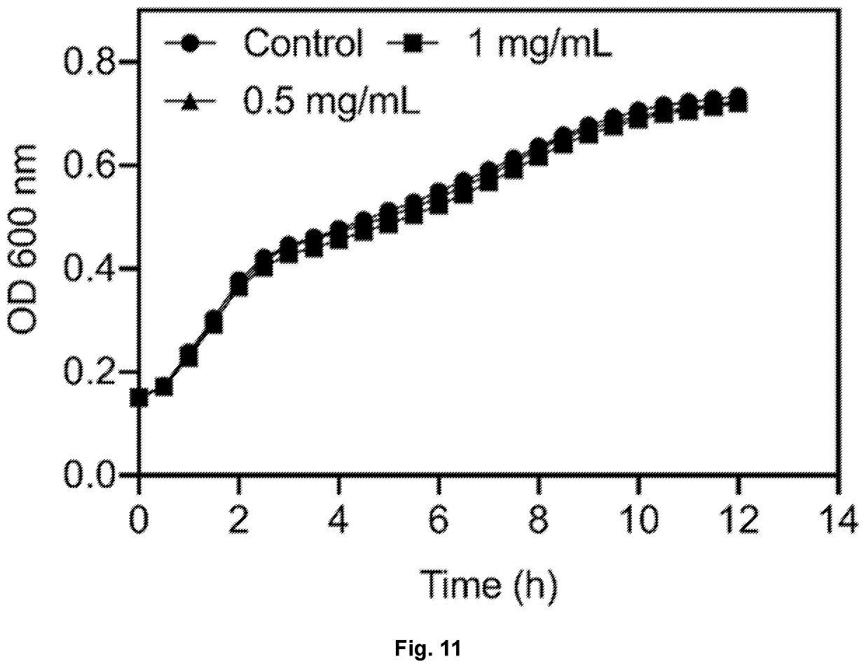

To determine the influence of the L100 layer on bacterial growth and viability, the growth curve of L100-TA-EcN was monitored. As shown in FIG. 1G, the growth of L100-TA-EcN was slightly slower than uncoated EcN in the initial 4 h but reached a similar rate afterward, which could be attributed to the gradual degradation of L100 in culture medium with neutral pH. Moreover, the co-culture of L100 copolymer and EcN did not display any inhibition of bacteria growth, demonstrating the negligible toxicity of L100 against EcN. See FIG. 11.

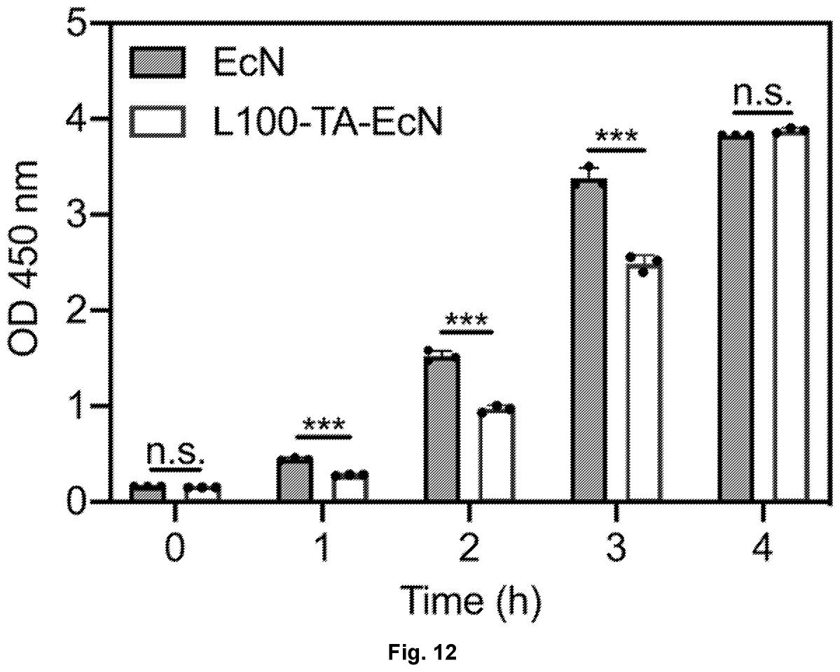

To investigate the impact of L100 on the proliferation of TA-EcN, a CCK-8 assay was performed. As shown in FIG. 12, the proliferation of TA-EcN was inhibited in the initial 3 h, which was consistent with the growth curve of L100-TA-EcN. The proliferation capability of L100-TA-EcN returned to a similar rate with EcN at 4 h, which is attributed to the complete degradation of the L100 layer (31).

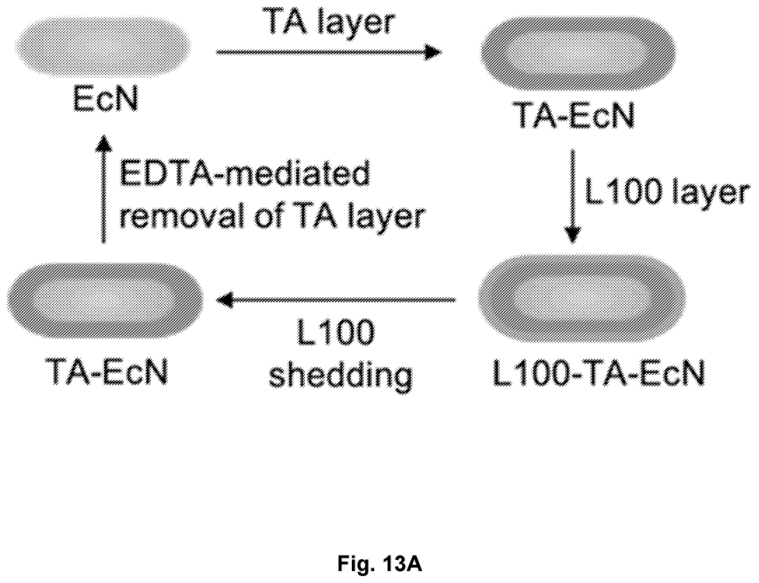

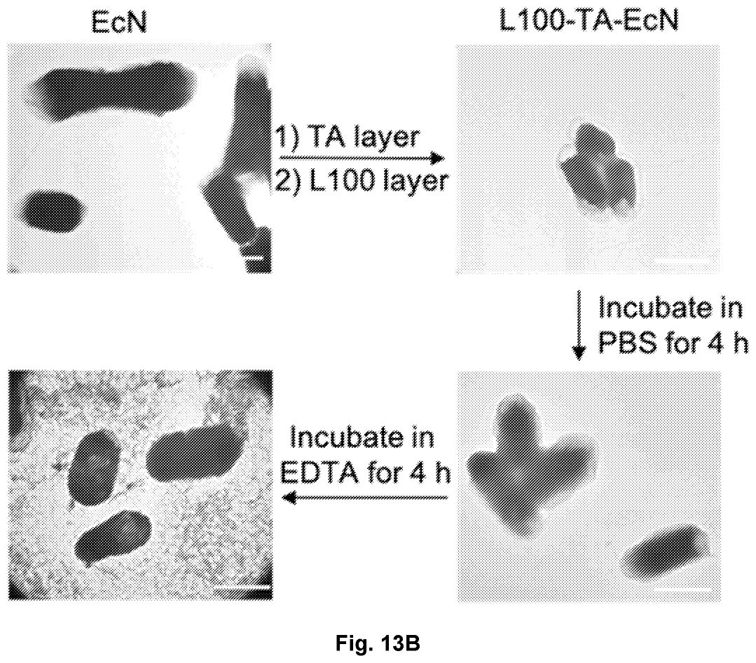

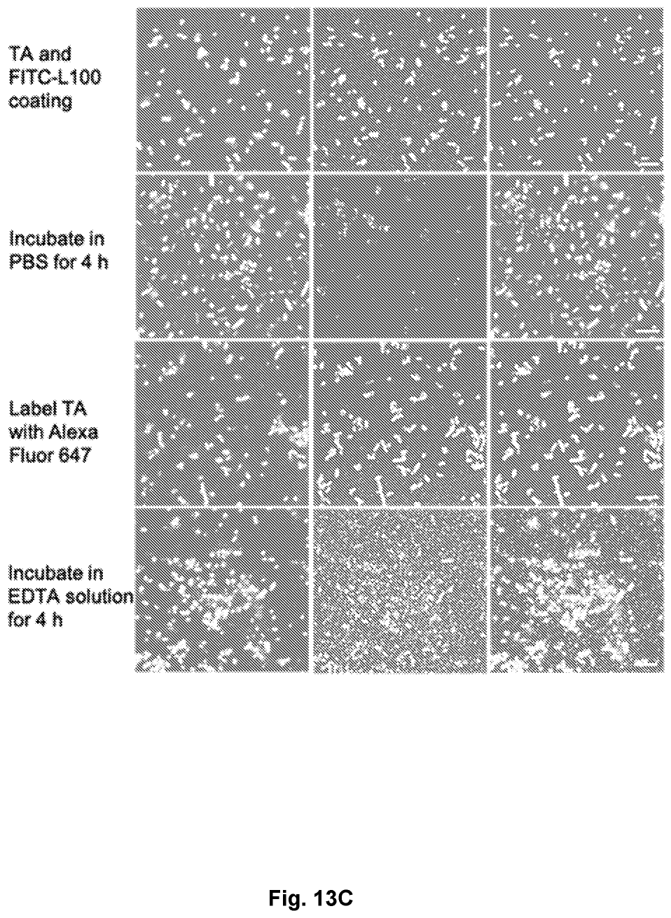

Next, the controlled degradation of the L100 copolymer layer and the tannin layer were investigated. After encapsulation of TA and L100 layers on the surface of EcN (see FIG. 13A), L100-TA-EcN was incubated in PBS (pH=7.4) for 4 h, followed by EDTA for 4 h. The TEM images revealed that the transparent outer layers on the surface of EcN became thinner when L100-TA-EcN was incubated in PBS for 4 h. Furthermore, there were no outer layers observed on the bacteria surface after continuous incubation in EDTA solution (FIG. 13B). This sequential degradation was further characterized with confocal microscopy, where FITC fluorescence from FITC-labelled L100-type copolymer diminished after 4 h incubation in PBS, and scattering Alexa fluor 647 fluorescence was observed due to dissociation of TA layer in EDTA solution. See FIG. 13C. Collectively, both copolymer and TA layers could be degraded on-demand to expose bacteria at the desired site.

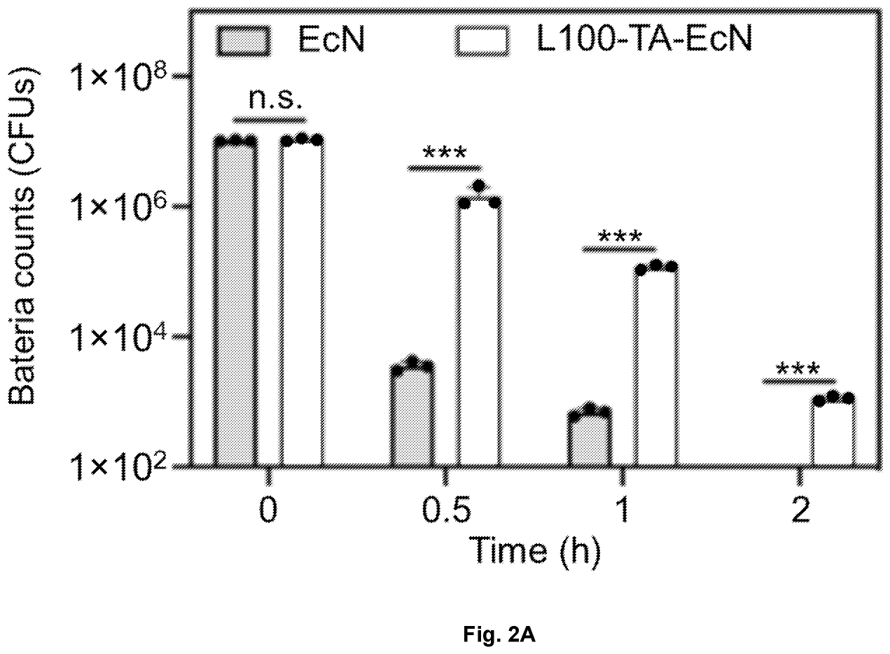

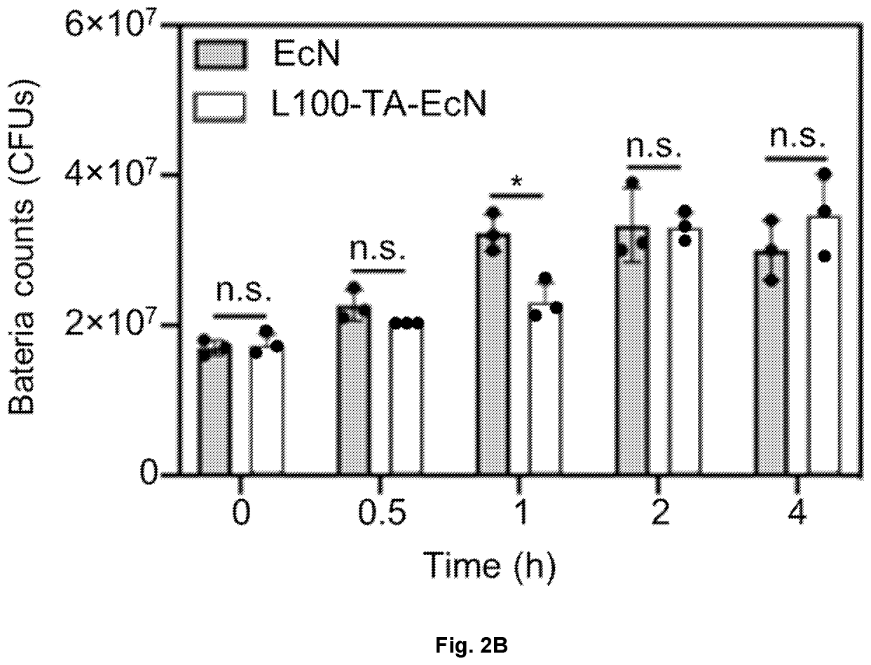

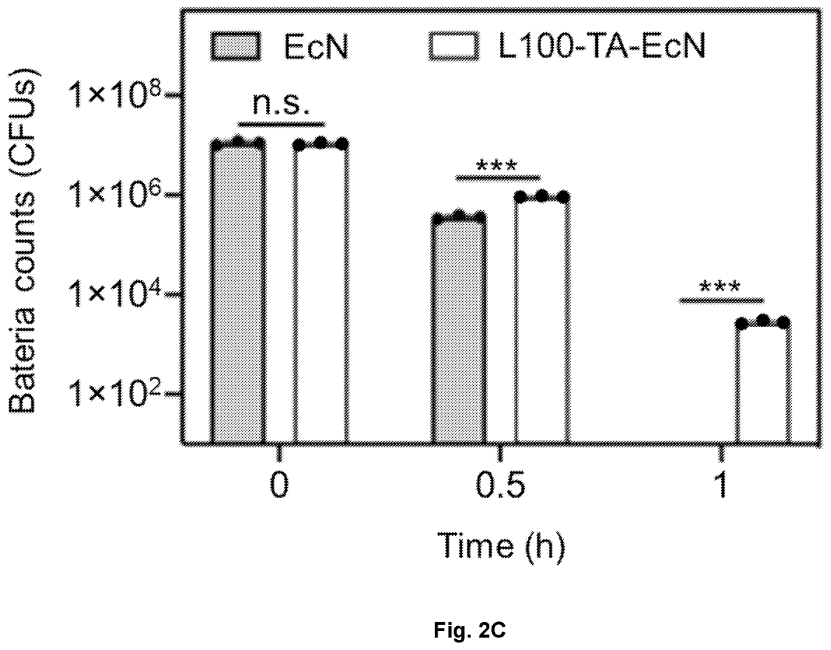

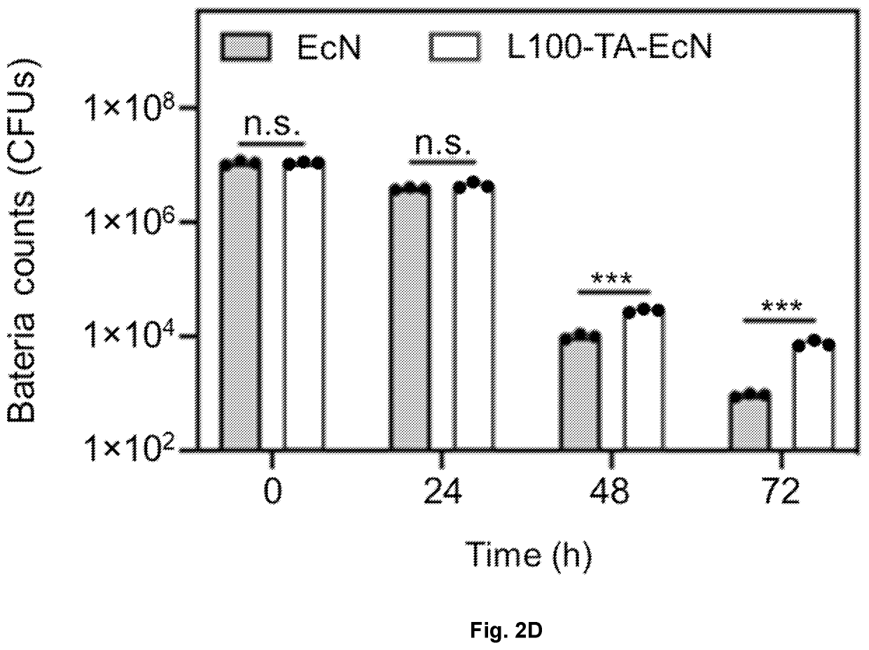

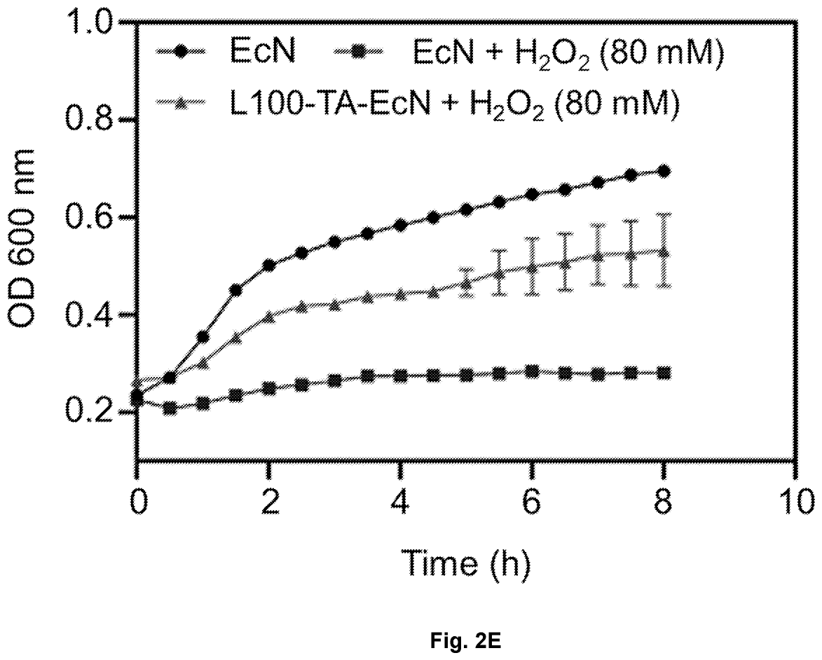

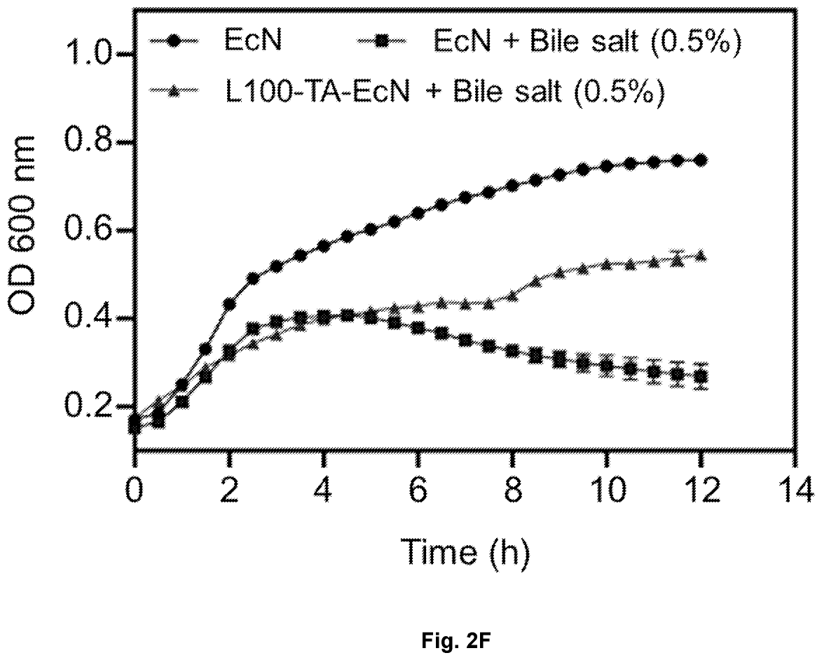

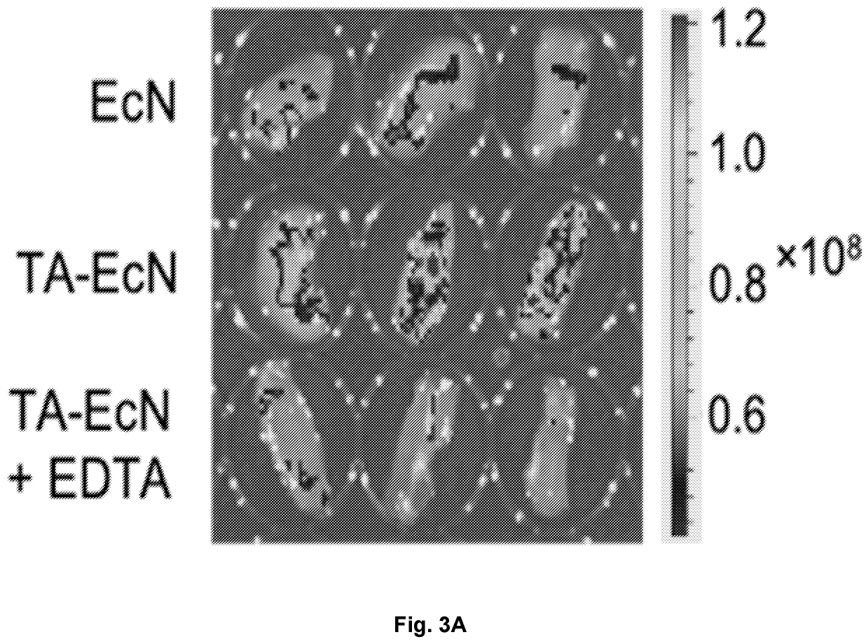

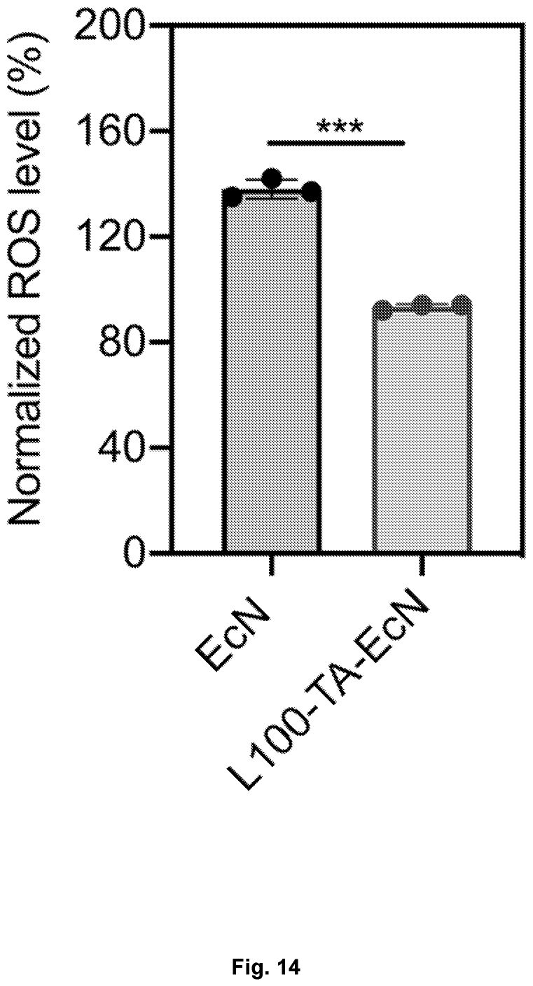

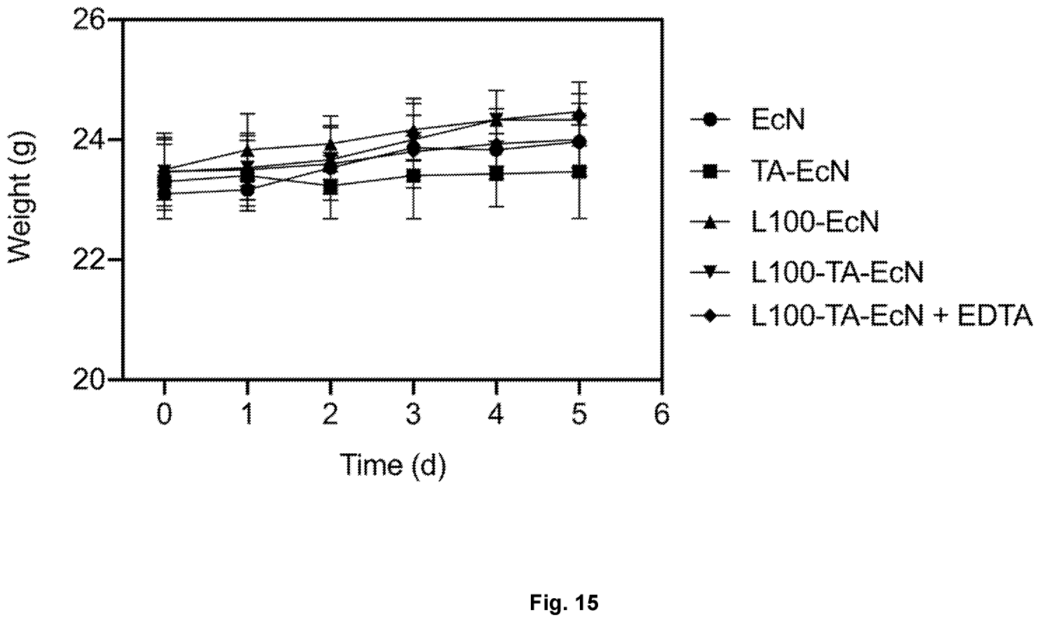

Next, whether the L100 and TA encapsulated EcN could resist environmental assaults was investigated. Firstly, L100-TA-EcN was subjected to simulated gastric fluid (SGF) supplemented with pepsin that mimics the acidic gastric environment. As shown in FIG. 2A, the L100-TA encapsulation significantly protected bacteria against SGF (pH=1.6). L100-TA-EcN displayed significantly higher viability than EcN after incubation with SGF for 0.5, 1, and 2 h. Notably, there were still more than 1×10³ live bacteria in the L100-TA-EcN group after 2 h incubation, whereas nearly 100% of EcN were dead. To investigate the viability of L100-TA-EcN in the intestinal fluid, a simulated intestinal fluid (SIF) containing trypsin was applied to evaluate the survival of L100-TA-EcN. As shown in FIG. 2B, the L100-TA-EcN reached a similar growth rate of EcN in SIF at 2 h. However, it required 4 h in LB medium due to pH-triggered degradation of the L100 layer, highlighting that L100-TA-EcN showed improved tolerance against the SIF solution. It was also found that L100-TA-EcN exhibited enhanced survival in strongly alkaline conditions (FIG. 2C) and antibiotics-containing solution (FIG. 2D) compared with uncoated EcN, indicating the protective capabilities of L100 and TA layers in the external environment. Given the anti-oxidization capabilities of TA (32), the viability of L100-TA-EcN in H₂O₂ containing medium was investigated. The EcN stimulated with 1 mM H₂O₂ generated a substantial amount of reactive oxygen species (ROS), significantly higher than that of L100-TA-EcN (FIG. 14). The superior anti-oxidization capability of L100-TA-EcN was further proved by a higher growth rate when compared to EcN in an LB medium containing H₂O₂ (80 mM) (FIG. 2E). Finally, the L100-TA-EcN exhibited improved tolerance against bile salt (0.5%) compared with uncoated EcN (FIG. 2F).

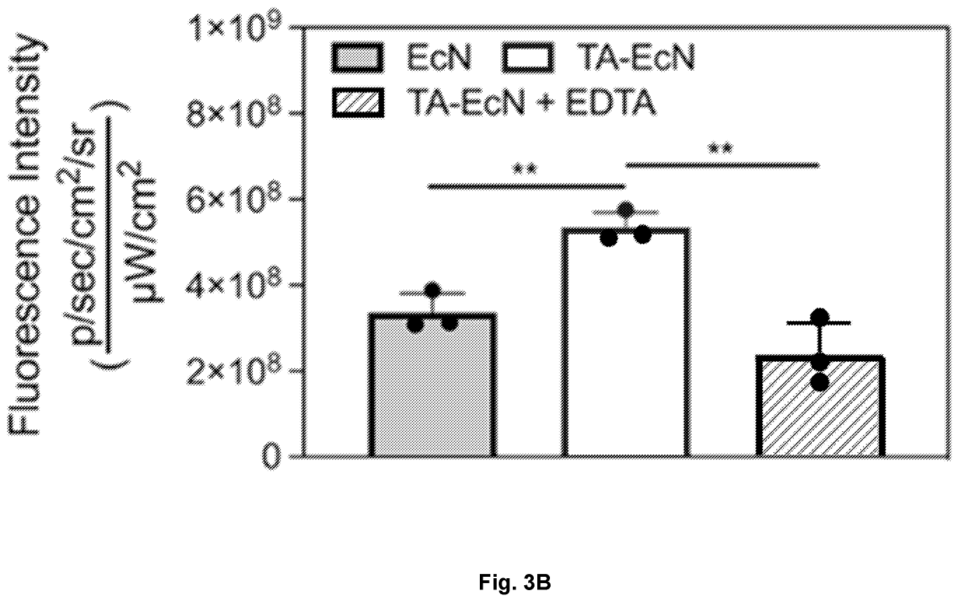

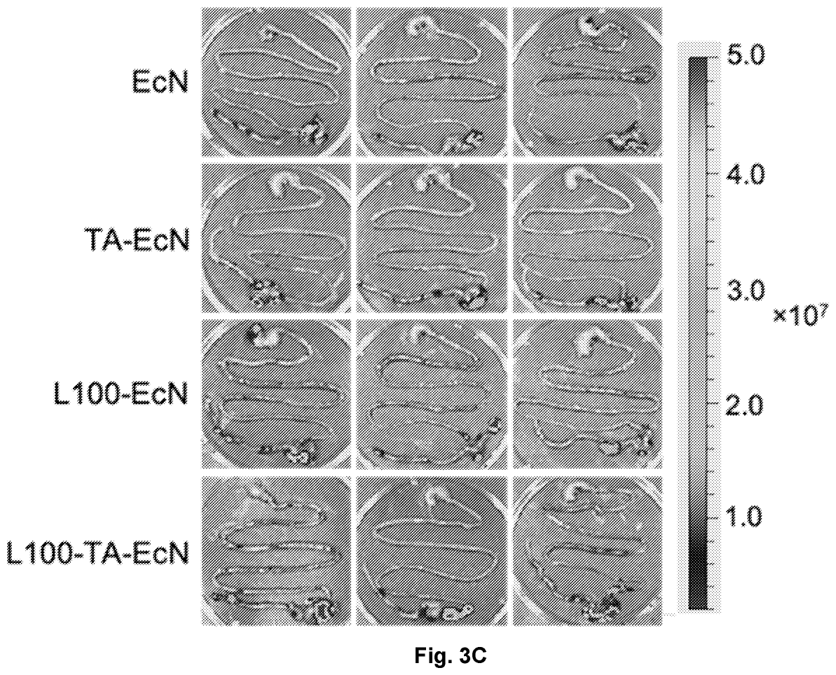

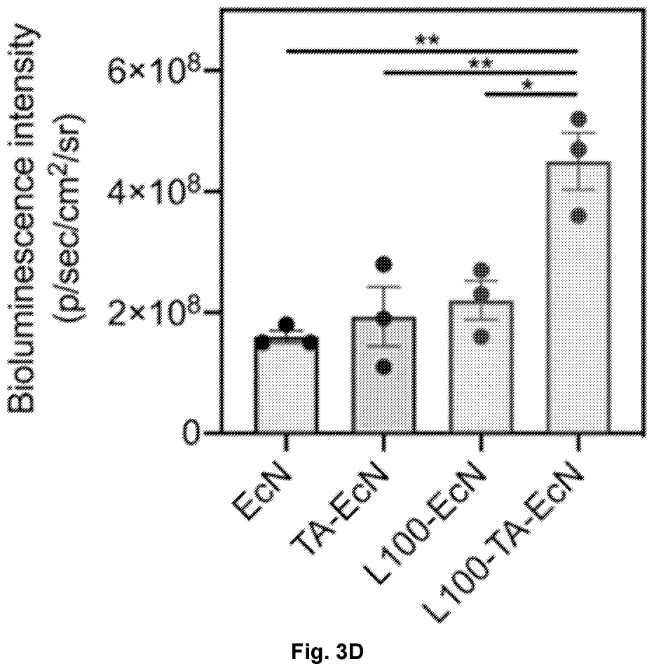

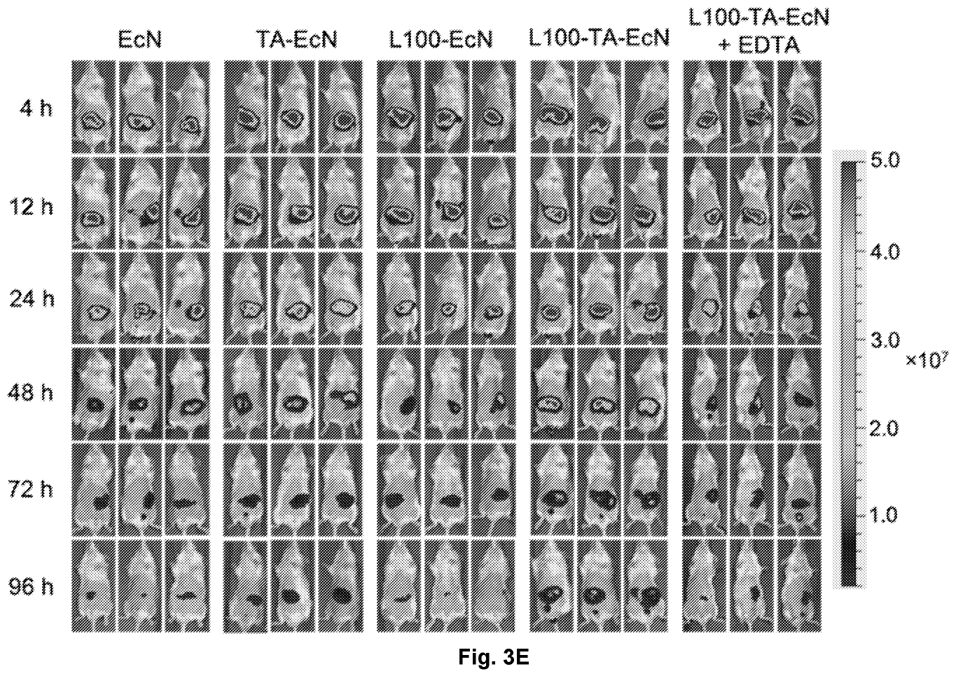

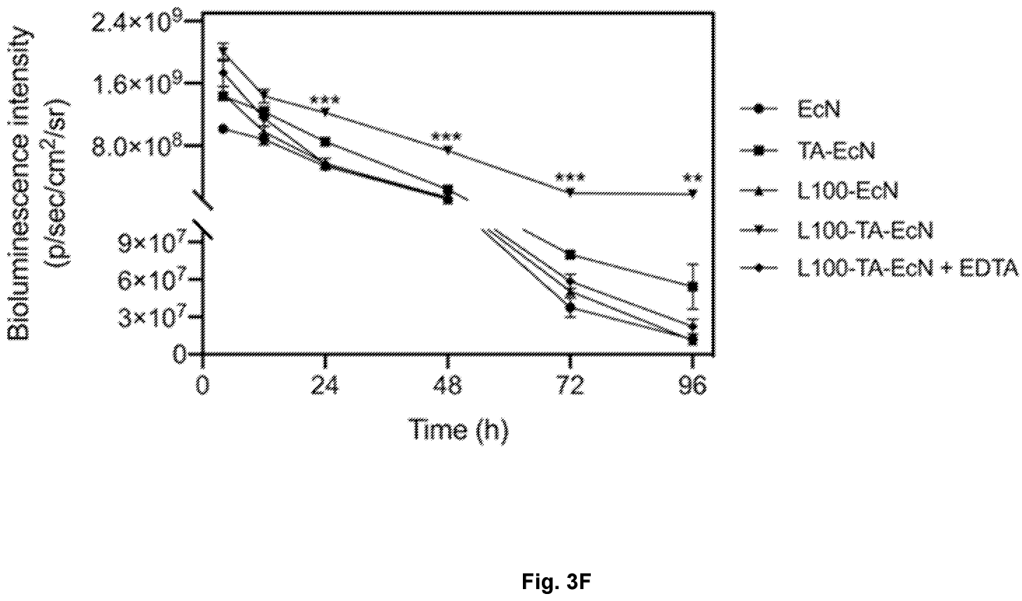

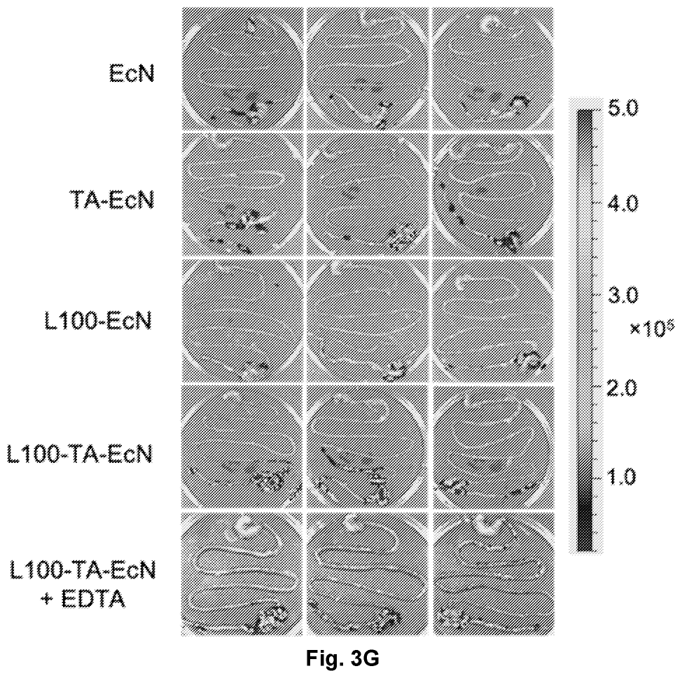

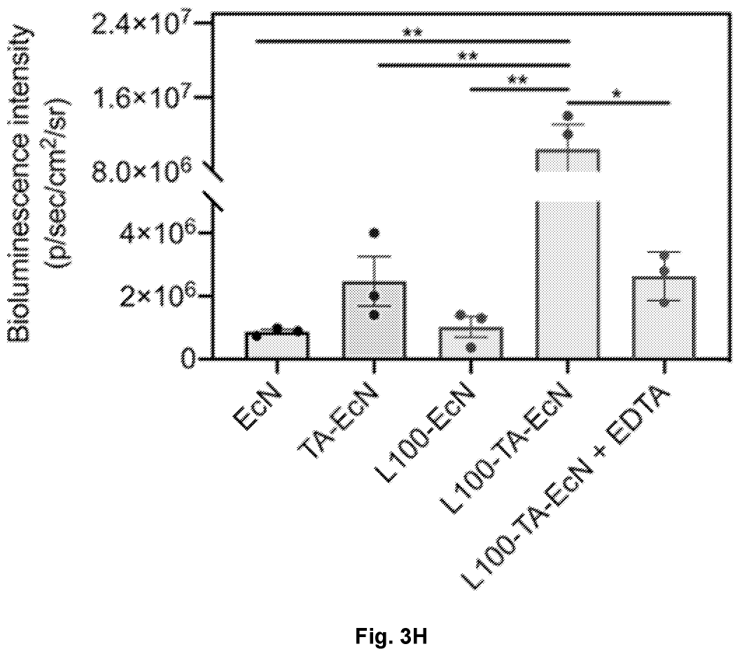

To investigate the mucoadhesive capability of the TA layer, rhodamine B-labeled EcN and TA-EcN were incubated with freshly collected mouse intestine tissues for 1 h. As shown in FIGS. 3A and 3B, the fluorescence intensity of the TA-EcN group was much higher than that of EcN, demonstrating that the TA layer could improve the adhesion capability of bacteria towards intestinal mucosa. However, the TA-EcN group treated with EDTA solution for 0.5 h displayed no significant difference in fluorescence intensity with the EcN group, suggesting that the addition of EDTA could dis-assemble the TA layer and disable mucosal binding.

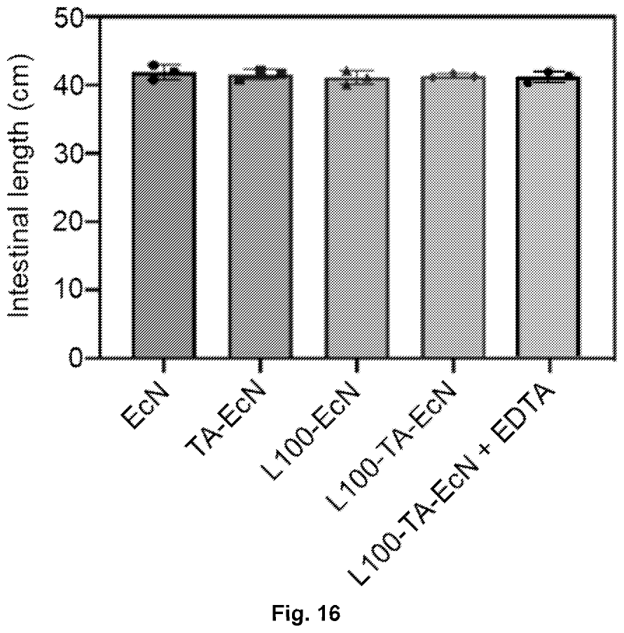

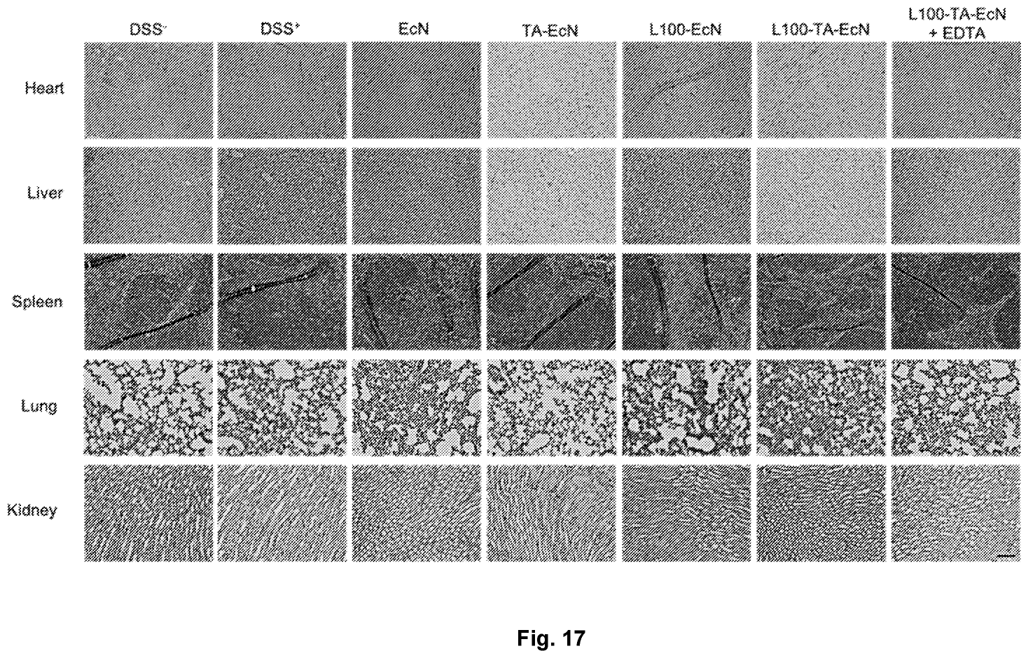

Next, in vivo intestine retention time of L100-TA-EcN was investigated. The EcN strains were electro-transformed with a plasmid of PAKgfpLux2 for in vivo bioluminescence-based imaging. As shown in FIGS. 3C and 3D, the mice intestines treated with L100-TA-EcN displayed the highest bioluminescence intensity among all treatment groups at 4 h, which was about 2.8-fold, 2.3-fold, 2.0-fold higher than EcN group, TA-EcN group, and L100-EcN group, respectively. There was no significant difference in bioluminescence intensity between TA-EcN and L100-EcN groups at 4 h, where both were slightly higher than the EcN group. Furthermore, the long-term characterization of mice treated with L100-TA-EcN showed prolonged retention time compared to other bacteria groups. See FIGS. 3E and 3F. The bioluminescence intensity of the L100-TA-EcN group was significantly higher than other bacteria groups in all detective timepoints, as evidenced by the slowest decay of bioluminescence signals compared to other groups. Notably, the L100-EcN group displayed a stronger bioluminescence signal than EcN group at 4 h, demonstrating the protection capability of the L100 layer against gastric acid. However, the bioluminescence decay kinetics between EcN and L100-EcN were similar after 12 h, which might be attributed to the limited mucosal adhesive effect. By contrast, the TA-EcN displayed a longer retention time than EcN and L100-EcN groups, highlighting that the TA layer enhances the adhesion of bacteria towards the intestine. Of note, with the addition of EDTA, the L100-TA-EcN group displayed a similar clearance time as the L100-EcN group due to the disassociation of the TA layer by EDTA. After 120 h, the mice were sacrificed, and the intestines were collected for IVIS®-brand imaging. (“Ivis” is a registered trademark of Xenogen Corporation, Hopkinton, Massachusetts, USA.) As shown in FIGS. 3G and 3H, the intestines treated with L100-TA-EcN were 11.5-fold, 4.0-fold, 10-fold, and 3.8-fold higher than EcN, TA-EcN, L100-EcN, and L100-TA-EcN+EDTA groups respectively, demonstrating the harsh environment resistance and mucoadhesive capability endowed by L100 and TA layers encapsulation. Moreover, there were no significant differences among TA-EcN, L100-EcN, and L100-TA-EcN+EDTA groups. The body weight (FIG. 15) and length of intestines (FIG. 16) showed no significant difference among all treatment groups during the time-course of treatment, demonstrating negligible side effects against mice.

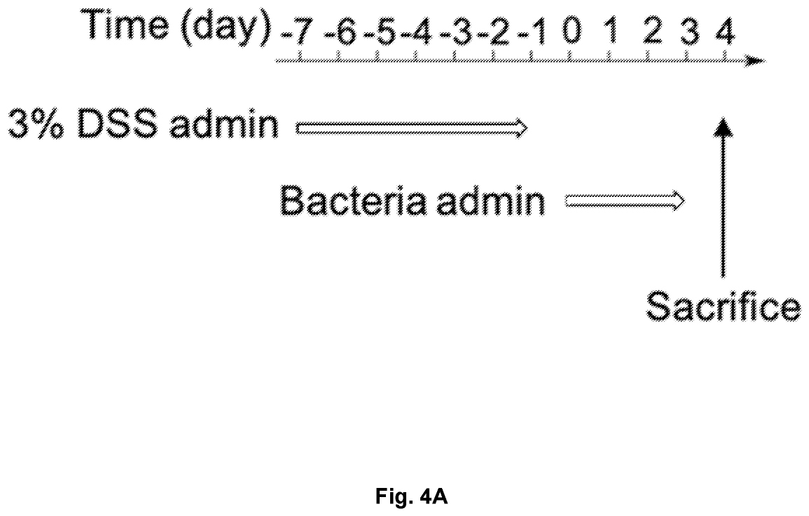

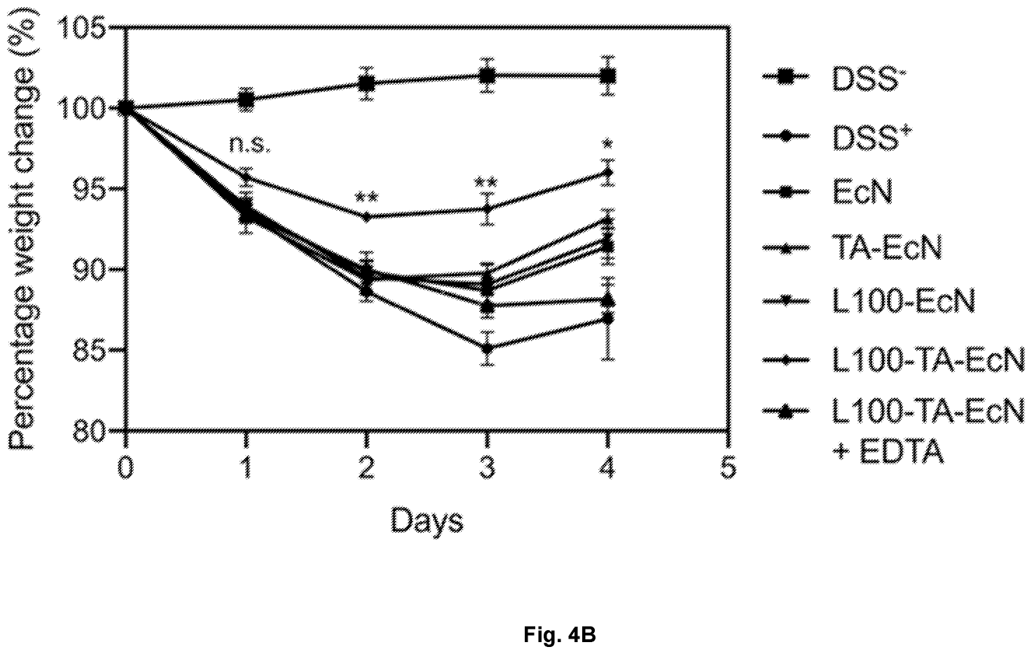

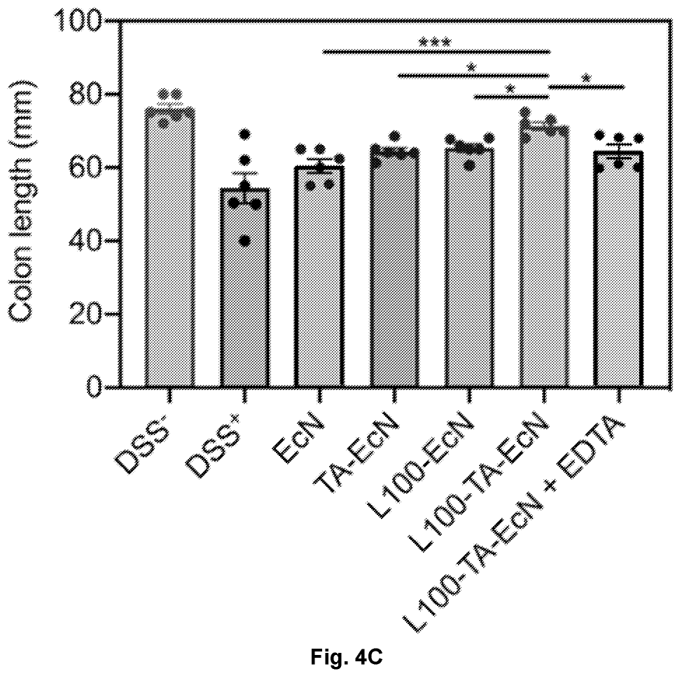

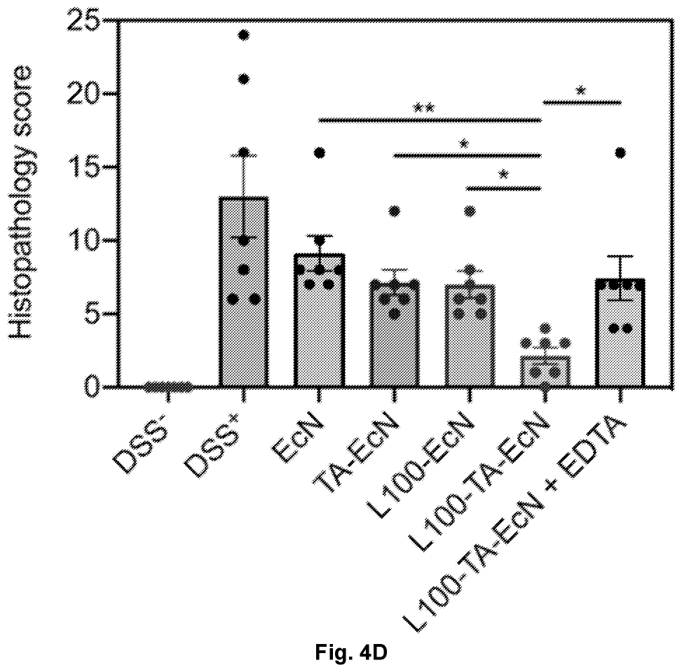

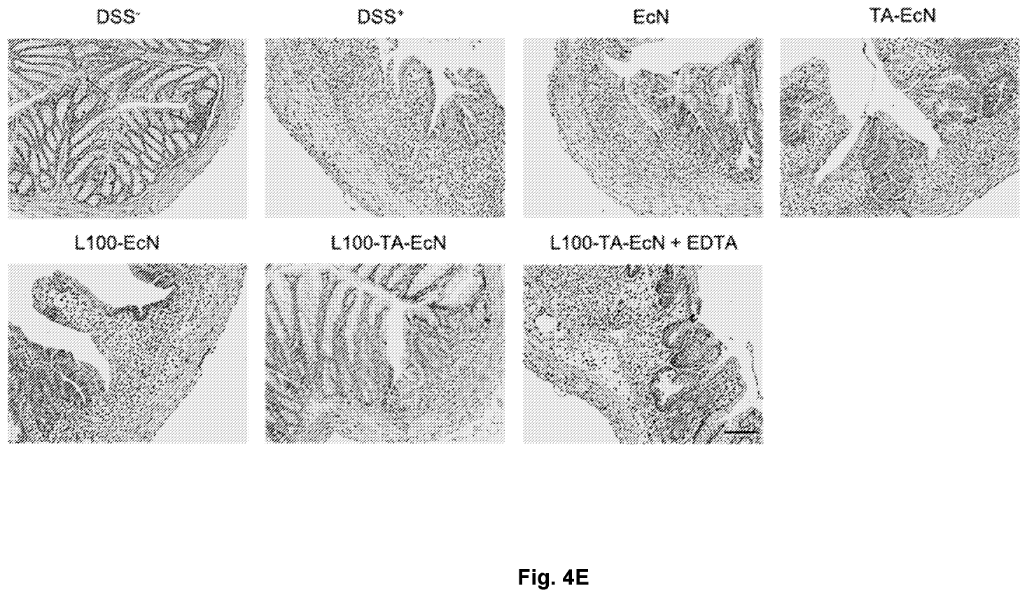

To validate the in vivo treatment efficacy of L100-TA-EcN against GI tract-related disease, the mouse colitis model was established by oral administration of dextran sulfate sodium (DSS) for a week (34). Afterward, the colitis-bearing mice were treated with PBS, EcN, TA-EcN, L100-EcN, L100-TA-EcN, L100-TA-EcN+EDTA (bacteria dose: 1×10⁸ CFU; EDTA: 100 mg/kg) for 4 consecutive days (FIG. 4A). The mice without DSS administration were set as the normal control. The treatment efficacy was monitored by changes in body weight that could intuitively reflect the severity of colitis. As shown in FIG. 4B, all the bacteria treatment groups displayed attenuated weight loss after day 2 when compared to the DSS⁺ group, demonstrating the beneficial efficacy of probiotics EcN. Notably, the mice treated with L100-TA-EcN displayed a substantially alleviated weight loss through 4-day time-course treatment, and the weight loss was significantly lower than all other treatment groups starting from day 2. By contrast, the addition of EDTA could significantly eliminate the superior treatment efficacy of L100-TA-EcN, as evidenced by continuous weight loss through the 4-day treatment and similar weight loss as DSS⁺ on day 4, which was attributed to the loss of mucoadhesive ability due to EDTA-induced disassembly of the TA-layer. Furthermore, colons of the treated mice were isolated, and colon length was measured to evaluate colon damage (35). As shown in FIG. 4C, the mice treated with L100-TA-EcN displayed a 6.2% reduction in colon length compared to that of DSS⁻ normal control, while the DSS⁺ group showed a 28% reduction, demonstrating the most prominent treatment efficacy of L100-TA-EcN against colitis. Moreover, the colon length in EcN, TA-EcN, L100-EcN and L100-TA-EcN+EDTA groups reduced 20.4%, 15.2%, 13.9% and 15.4% compared to DSS⁻ normal control, respectively. The treatment efficacy was then assessed by evaluating colon damage through histological analysis. A histological scoring system related to inflammatory severity, depth of injury, crypts damages, and percent involvement was applied to quantitatively examine the colon conditions. See Table 2 in the Examples section (36). As shown in FIG. 4D, histopathology scores of the L100-TA-EcN treatment group were significantly lower than all other DSS treatment groups, displaying a 6.1-fold, 4.3-fold, 3.3-fold, and 3.3-fold lower than DSS+, EcN, TA-EcN, and L100-EcN groups, respectively. Of note, the addition of EDTA significantly compromised the treatment efficacy of L100-TA-EcN, showing a 3.5-fold higher histopathology score than L100-TA-EcN. Furthermore, the representative histology images showed a complete loss of crypt, goblet cell depletion and immune cell infiltration in the DSS⁺ group, whereas substantial improvements were found in all bacteria treatment groups. The administration of L100-TA-EcN showed an almost intact epithelium layer and negligible inflammatory cell infiltration, indicating the superior efficacy of treatment against colitis (FIG. 4E). Moreover, histological analysis of major organs (FIG. 17), including heart, liver, spleen, lung and kidney, along with the complete blood count (Table 1 in the Examples), indicated no significant side effects caused by all treatment groups.

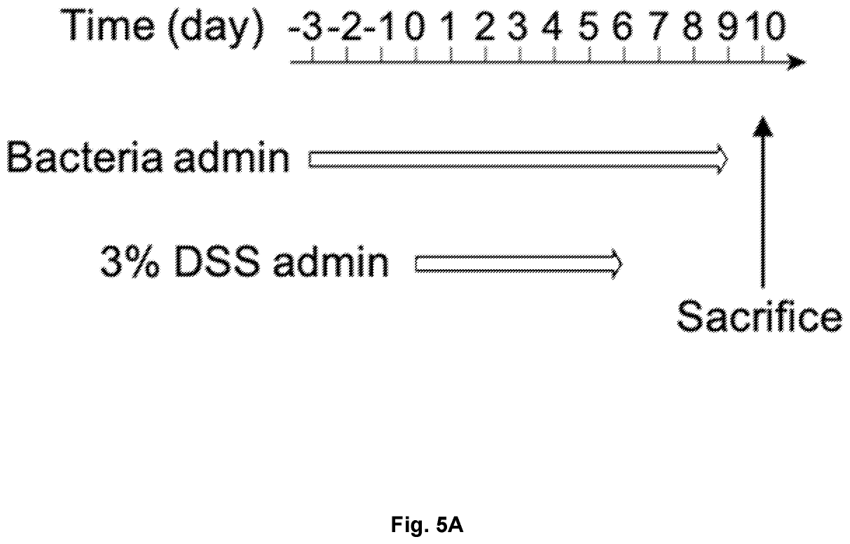

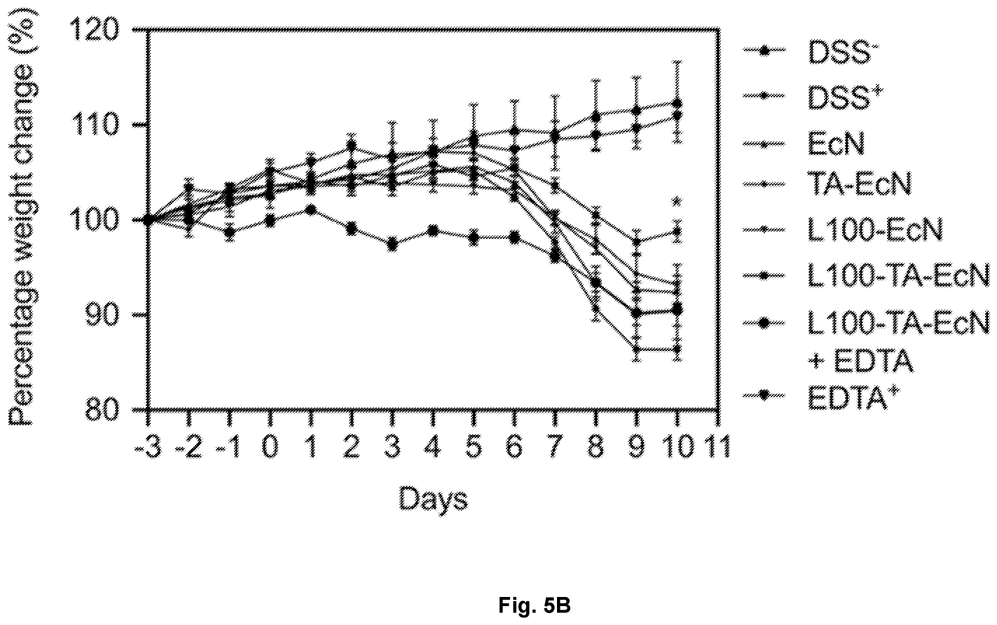

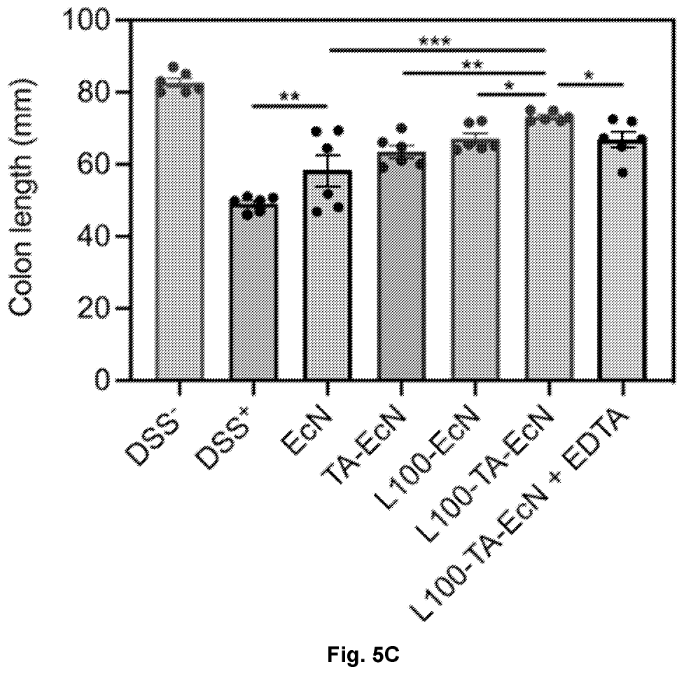

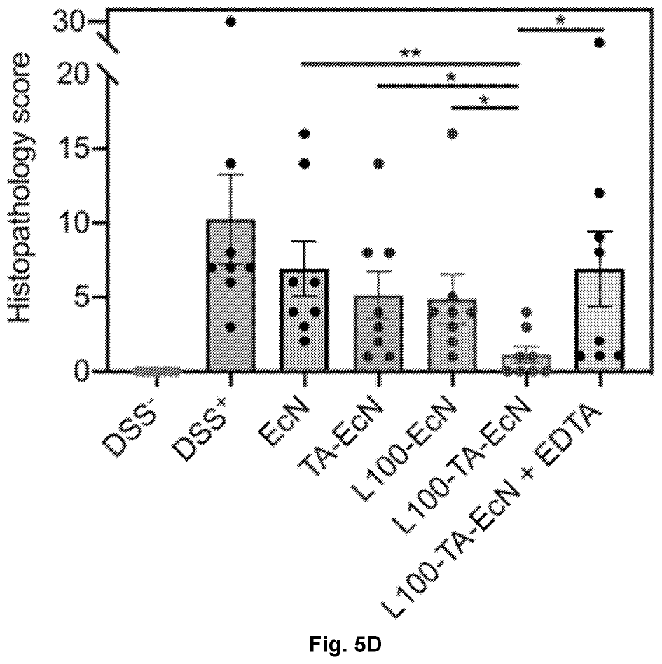

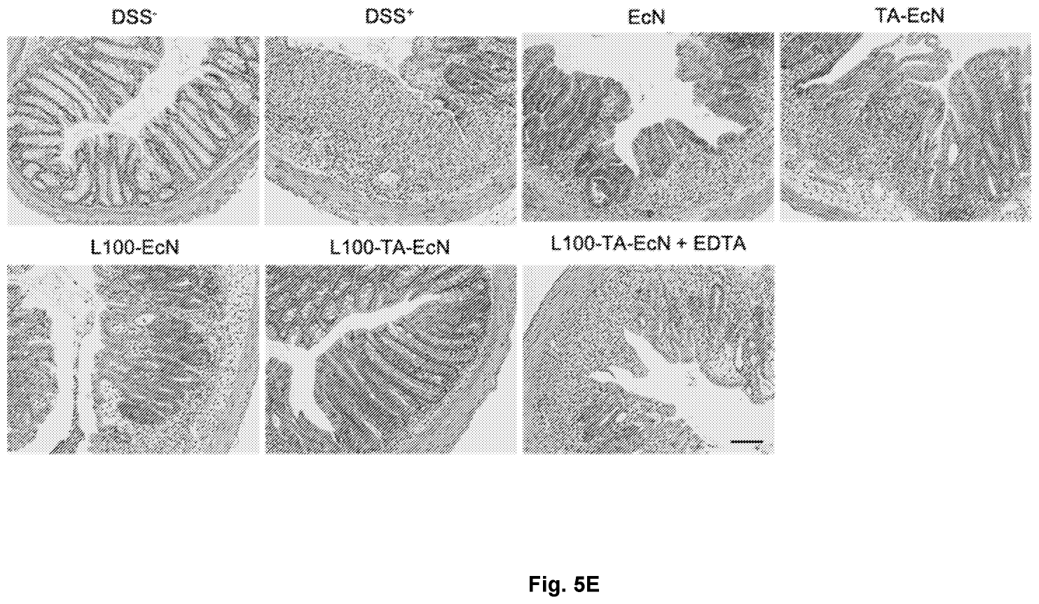

To investigate whether the pre-treatment of L100-TA-EcN could exhibit prophylactic efficacy to protect mice from colitis, various EcN-based treatment formulations were orally administered to mice for 3 days. Afterward, the mice were fed with DSS together with all bacteria formulations for 7 days, and only bacteria formulations for a continued 3-day recovery period. See FIG. 5A. Mice treated with L100-TA-EcN exhibited weight loss from Day 6, whereas other groups showed weight loss from Day 5. See FIG. 5B. Furthermore, the mice in the L100-TA-EcN group showed significantly ameliorated weight loss from day 7 compared to all other groups. Notably, from day 9, the mice's weight began to recover in the L100-TA-EcN group, while other groups exhibited continuous weight loss. The mice only treated with EDTA displayed a weight increase through the 2-week time-course treatment, indicating that no significant side effects were induced by EDTA administration. Furthermore, as shown in FIG. 5C, the colon length in the L100-TA-EcN group was significantly longer than that in other DSS treatment groups. The histology score for the L100-TA-EcN group was the lowest among all groups that received DSS treatment, indicating its superior protective effect in preventing morbidity of colitis (FIG. 5D). In addition, hematoxylin and eosin (H&E) staining revealed the lowest colon damage for the L100-TA-EcN group compared to all other DSS treatment groups. (FIG. 5E).

In summary, disclosed herein is a double-layer coating strategy for effective oral delivery of probiotics in which one layer protects probiotics from environmental stress, and the other layer offers robust adhesion of probiotics towards mucosa. This delivery approach enhances viability of probiotics after oral administration and extends retention time of bacteria in the GI tract. By taking advantage of the pH-responsive degradation of L100-type copolymers, L100-TA-EcN could resist the acidic environment in the stomach, while selectively releasing TA-EcN in the GI tract upon return to physiological pH level. Furthermore, the exposed TA-EcN after L100 disassociation could display a strong mucosal adhesion that can increase the retention of EcN. In a DSS-induced mouse colitis model, L100-TA-EcN displayed superior prophylactic and treatment efficacy against colitis and mitigated weight loss in mice. Furthermore, to eliminate any potential toxicity, EDTA was applied to remove the TA layer lining on the mucosal epithelial layer that may potentially impact nutrient and drug absorption. This double-layer coating strategy can be adapted to encapsulate other living cells for specific disease treatment, where cell protection before release and spatiotemporal cell delivery are critical.

### Examples

The following examples are included herein to provide a more complete description of the methods and compositions of matter disclosed herein. The examples are not intended to limit the scope of the claims in any fashion.

Materials. The following chemicals and biologicals were used in this study: Tannic acid (TA, Sigma), iron (III) chloride hexahydrate (FeCl₃·6H₂O, Sigma Chemical, St. Louis, Missouri, USA), 3-(N-morpholino)propane sulfonic acid (MOPS, Sigma), Calcium chloride (CaCl₂), Sigma), “EUDRAGIT”® L100-brand polymers (Evonik Industries, Parisppany, New Jersey, USA), hydrochloric acid (HCl, 37%, Lab Chem, Zelienople, Pennsylvania, USA), Ethylenediaminetetraacetic acid (EDTA, Sigma), N-Hydroxysuccinimide (NHS, Alfa Aesar, Ward Hill, Massachusetts, USA), Potassium phosphate (KH₂PO₄, Sigma), Sodium hydroxide (NaOH, Alfa Aesar), Trypsin from porcine pancreas (Sigma), Pepsin powder (Fisher Scientific, Waltham, Massachusetts, USA), Bile salts (Sigma), Ampicillin (Sigma), Dextran sulfate sodium salt (DSS, MW 40,000, Alfa Aesar), LB broth (Fisher Scientific), Agar (Fisher Scientific), Glycerin (Ward's Science, Rochester, NY, USA), hydrogen peroxide solution (H₂O₂, Sigma), Sodium chloride (NaCl, Fisher Scientific), Alexa Fluor® 647 conjugated albumin from bovine serum (BSA-Alexa Fluor® 647, Life Technologies, Waltham, Massachusetts, USA), Hoechst 33342 trihydrochloride (Life Technologies), Cell counting kit-8 (CCK-8, APExBIO Technology, LLC, Houston, Texas, USA), 2′,7′-Dichlorofluorescin diacetate (DCFH-DA, Sigma), Isopropyl β-D-1-thiogalactopyranoside (IPTG, Chem-Impex, Wood Dale, Illinois, USA). Luria-Bertani (LB) liquid media was prepared with 25 g of LB broth in 1 L of deionized (DI) water and used after autoclaved. LB agar plates were prepared on dishes with 20 mL of LB agar solution (25 g of LB broth and 12 g of agar in 1 L of DI water). Simulated gastric fluid (SGF) and simulated intestinal fluid (SIF) were prepared as described in the United States Pharmacopoeia. Briefly, SGF was prepared by dissolving 2.0 g of NaCl and 3.2 g of pepsin in 1 L of DI water, and pH was adjusted to 1.6 with HCl. The SGF was filtered by 0.22 μm membrane before usage. SIF was prepared by dissolving 6.8 g of KH₂PO₄ and 10 g of trypsin in 1 L of DI water, and pH was adjusted to 6.8 with NaOH. The SIF was filtered by 0.22 μm membrane before usage.

Strain and culture conditions. The probiotic of Escherichia coli Nissle 1917 cells (EcN; MUTAFLOR®-brand probiotic) was purchased from Pharma-Zentrale GmbH, Herdecke, Germany. The EcN cells were cultured on an LB agar plate (1.5% agar). Before each experiment, the cells were cultured in liquid LB medium overnight at the shaking speed of 225 rpm at 37° C. See Sonnenborn, U. and Schulze J., The non-pathogenic Escherichia coli strain Nissle 1917—features of a versatile probiotic Microb Ecol Health Dis (2009) 21, 122-58.

Encapsulation of EcN with TA. The EcN cells were picked from an LB agar plate and cultured in LB medium at 37° C. overnight. Then, the EcN cells were washed with DI water twice and resuspended in 490 μL of DI water. Afterwards, 5 μL of TA solution (40 mg/mL) and 5 μL of FeCl₃ solution (10 mg/mL) were added to the suspension successively, and the mixture was gently vortexed for 20 seconds. After that, 0.5 mL of MOPS buffer (20 mM, pH 7.4) was added to stabilize pH, resulting in the formation of a stable Ta-Fe layer on the surface of EcN. The formed TA-EcN cells were collected by centrifugation after being washed with DI water twice to remove residual TA and FeCl₃.

Encapsulation of TA-EcN with enteric L100. The formed TA-EcN cells were resuspended in 0.9 mL of PBS solution containing 12.5 mM of CaCl₂) and vortexed for 5 min. 100 μL of L100 solution (1 mg/mL) was added to the suspension before vortexing for 5 min. The pH of resulting suspension was adjusted to 5.5 to generate a stable L100 layer on the surface of TA-EcN, and L100-TA-EcN cells were collected via centrifugation.

Characterization of TA-EcN and L100-TA-EcN. The morphologies of TA-EcN and L100-TA-EcN were visualized by transmission electron microscopy (TEM, FEI Tecnai T12). Briefly, a drop of bacteria solution was deposited onto a carbon-coated copper grid. The samples were then washed with DI water, entirely dried in the air, and imaged by TEM. The encapsulation of EcN in TA and L100 layers were also characterized by laser scanning confocal microscopy (LSCM, Nikon AIRS). Briefly, EcN cells were incubated with Hoechst 33342 at a final concentration of 5 μg/mL for 10 min in the dark to label EcN cells. After washing with DI water to remove the residual dye, Hoechst 33342 labeled EcN was encapsulated with the TA layer to prepare TA-EcN. TA-EcN was then mixed with BSA-Alexa fluor 647 solution and incubated for 30 min to label the TA layer. Afterward, FITC-labeled L100 was coated on the TA-EcN, and L100-TA-EcN was subjected to LSCM for observation. Moreover, the particle sizes and zeta potentials of EcN, TA-EcN and L100-TA-EcN were determined by dynamic light scattering (DLS).

Measurement of growth curves of EcN, TA-EcN, and L100-TA-EcN. After encapsulating EcN with TA and L100 layers, the bacteria were diluted and inoculated into a 96-well plate with an OD₆₀₀ value˜0.15 and incubated at 37° C. with gentle shaking. The OD₆₀₀ values were monitored for 12 h at 0.5 h intervals by a microplate reader (Infinite M Plex, Tecan). Uncoated EcN was used as a control.

Cell viability assay. Cell viability was examined by a cell counting kit-8 (CCK-8) assay according to the manufacturer's instructions. Briefly, 100 μL of EcN, TA-EcN, and L100-TA-EcN in LB medium were separately seeded into a 96-well plate with an initial OD₆₀₀ value˜0.15. Afterward, 10 μL of CCK-8 solution was added into each well and cultured at 37° C. The OD values were recorded at 450 nm for 4 h at 1 h intervals by a microplate reader.

Degradation of L100 and TA layers. After sequentially coating the L100 and TA layers on EcN, L100-TA-EcN cells were suspended in PBS solution, and incubated at 37° C. with gentle shaking for 4 h. Afterward, the resulted TA-EcN cells were washed twice with DI water and imaged by TEM. The TA-EcN was continued to incubate in EDTA solution (1 mg/mL) at 37° C. for 4 h, and then imaged by TEM after washing with DI water. The degradation of L100 and TA layers was also observed via confocal microscopy. Briefly, EcN cells were encapsulated with TA layer and FITC-labeled L100 polymer. After incubation in PBS for 4 h at 37° C., the L100 layer on TA-EcN was imaged by confocal microscopy. The resulted TA-EcN was further incubated with BSA-Alexa fluor 647 for 30 min. Afterward, Alexa fluor 647-labeled TA-EcN was suspended in EDTA solution for 4 h, and the TA-EcN was subjected to confocal microscopy for characterization.

Resistance assay. Equal amounts of EcN and L100-TA-EcN were separately subjected to SGF (pH=1.6) supplemented with pepsin (0.32%), SIF (pH 6.8) supplemented with trypsin (10 mg/mL), strong alkali solution (pH=11.0) and ampicillin solution (0.5 mg/mL), and incubated at a shaking speed of 225 rpm at 37° C. At predetermined time points, 50 μL of each sample was taken, washed with DI water, and spread on LB agar plates in sequential 10-fold dilutions. The colonies were counted after 24 h of incubation at 37° C. For resistance against H₂O₂ stimulation and bile salt, EcN and L100-TA-EcN were incubated in LB medium containing H₂O₂ (80 mM) or bile salt (0.5%), and the growth curves were monitored at OD_{600 nm} by a microplate reader.

Detection of ROS. ROS generation was quantified by 2′-7′-dichlorodihydrofluorescein diacetate (DCFH-DA) staining. DCFH-DA can be hydrolyzed by esterase to DCFH intracellularly and further be oxidized to the fluorescent DCF by superoxide. Briefly, EcN and L100-TA-EcN cells were separately incubated with H₂O₂ (1 mM) solution at 37° C. for 1 h. Then, the cells were washed with DI water twice to remove residual H₂O₂. Finally, DCFH-DA solution was added at a final concentration of 40 μg/mL and incubated for 30 min in the dark. After washing with DI water to remove residual DCFH-DA, the fluorescence intensity of cells was detected by a microplate reader.

Mucoadhesive evaluation in vitro. Rhodamine B-NHS was incubated with EcN for 1 h with slow stirring to label bacteria. The rhodamine-labeled bacteria were washed with PBS three times to remove the residual dye. After encapsulation with TA, the bacteria were suspended in DI water. Freshly isolated mice intestine was cleaned and sectioned. Rhodamine B-labeled EcN and TA-EcN in DI water were incubated with the mice intestines in a 24-well plate for 1 h at 37° C. The intestines were then washed with DI water for 20 min before being imaged using an IVIS®-brand imager. For the EDTA group, the intestines treated with TA-EcN were further incubated with EDTA solution (1 mg/mL) for 20 min, washed with DI water and imaged by an IVIS®-brand imager (Perkin Elmer).

Electrotransformation of EcN with PAKgfpLux2. The EcN cells were electrotransformed with the plasmid of PAKgfpLux2 for monitoring cell distribution in vivo. Briefly, 40 μL of the EcN cells (10⁹ CFU) were mixed with 1 μL of the plasmid of PAKgfpLux2 (available commercially from Addgene, Watertown, Massachusetts, USA) at a final concentration of 1 μg/mL and incubated on ice for 1 min. The mixture of cells and plasmid was transferred to a cold electroporation cuvette (0.1 cm) and tapped to the bottom. The cuvette was placed into a chamber and pulsed once (1.8 kV, 4 milliseconds, MicroPulser, BIO-RAD, Hercules, California, USA). Afterward, the cuvette was removed from the chamber and 1 mL of LB medium was added immediately. The cell suspension was then transferred to a tube and incubated at 37° C. After 1 h, the suspension was spread on an antibiotic selective LB agar plate (100 μg/mL of ampicillin) and incubated overnight.

Evaluate the adhesive effect of L100-TA-EcN in vivo. To further evaluate the adhesive effect of L100-TA-EcN in vivo, the luciferase-expressed EcN cells were encapsulated with TA and L100 layers and orally administered to mice, and the distribution of EcN cells was monitored through bioluminescence signals via an IVIS®-brand imager. All the animal studies strictly followed the animal protocols approved by the Institutional Animal Care and Use Committee at the University of Wisconsin-Madison. Briefly, mice (male, aged 5-6 weeks) were randomly assigned into six groups (n=3 in each group) and administered with PBS, EcN, TA-EcN, L100-EcN, L100-TA-EcN, and L100-TA-EcN followed EDTA treatment at the EcN dose of 1×10⁹ CFU, respectively. Mice were fasted for 18 h prior to experiments and received IPTG (5 g/L) and ampicillin (1 g/L) via drinking water. Moreover, the bioluminescence of the cells was also induced by IPTG (1 mM) before administration. Mice were given either 100 μL of PBS or bacterial suspension via oral gavage. Afterward, the mice were returned to normal chow with IPTG and ampicillin in drinking water. At 4 h, 12 h, day 1, day 2, day 3, and day 4, the mice were imaged under anesthesia using an IVIS. For the EDTA treatment group, after 4 h and 12 h administration of L100-TA-EcN, 100 μL of EDTA (100 mg/kg) solution was administered via oral gavage, and imaged by an IVIS. The mice were sacrificed on day 5, and the GI tract was collected for IVIS®-brand imaging. The mice's body weight was recorded daily, and the length of intestines was measured.

Prophylactic efficacy of L100-TA-EcN against DSS-induced colitis. The prophylactic efficacy of L100-TA-EcN was evaluated in a DSS-induced mice colitis model. Briefly, mice (male, aged 5-6 weeks) were randomly divided to eight groups: PBS-DSS⁻, PBS-DSS⁺, EDTA⁺, EcN, TA-EcN, L100-EcN, L100-TA-EcN, and L100-TA-EcN followed by EDTA treatment (n=6). The mice were fed with normal chow and administered either PBS, EDTA (100 mg/kg), or bacterial suspensions (bacteria dose: 1×10⁸ CFU) based on their group assignments via oral gavage daily throughout the experimental period. Three days after bacteria administration, DSS (3%) was added to the drinking water to induce colitis except for PBS-DSS⁻ group for 7 days. After DSS removal, all mice were given normal drinking water until day 13 before sacrificed. On day 13, the mice intestines were harvested, and the colon length was measured. The colon tissues were fixed in 4% paraformaldehyde for histological analysis. Mouse body weight was recorded daily.

Therapeutic efficacy of L100-TA-EcN against DSS-induced colitis. To evaluate the therapeutic efficacy of L100-TA-EcN against the DSS-induced colitis, the colitis-bearing mouse model was established as described above. Briefly, the mice were given drinking water containing 3% of DSS for 7 days first to induce colitis. Afterward, the mice were given normal drinking water and bacterial suspensions (PBS, EcN, TA-EcN, L100-EcN, L100-TA-EcN, and L100-TA-EcN+EDTA; bacteria dose: 1×10⁸ CFU; EDTA: 100 mg/kg) were administered for another 4 days. At day 11, the mice were sacrificed, and the blood was collected for complete blood count by the hematology analyzer (Abaxis VetScan HM5). Blood count results are presented in Table 1. The colon length was measured. The colon tissues and major organs, including heart, liver, spleen, lung and kidney, were harvested and fixed in 4% paraformaldehyde for histological analysis. The mouse body weight was recorded daily.

TABLE 1
Complete blood counts of mice in different groups.

| | WBC | LYM | MON | NEU | RBC | HGB |
|---|---|---|---|---|---|---|
| | (10⁹/L) | (10⁹/L) | (10⁹/L) | (10⁹/L) | (10¹²/L) | (g/dL) |
| Reference range | 3.48-14.03 | 2.22-9.83 | 0.21-1.25 | 0.58-3.83 | 6.93-12.24 | 12.6-20.5 |
| Healthy control | 4.19 ± 0.59 | 3.39 ± 0.49 | 0.17 ± 0.02 | 0.45 ± 0.18 | 9.67 ± 0.27 | 14.9 ± 0.44 |
| DSS⁺ | 4.55 ± 2.05 | 3.67 ± 1.96 | 0.20 ± 0.05 | 0.68 ± 0.51 | 10.0 ± 0.40 | 14.7 ± 0.77 |
| EcN | 3.92 ± 1.84 | 3.20 ± 1.50 | 0.21 ± 0.06 | 0.63 ± 0.41 | 9.93 ± 0.26 | 14.8 ± 0.50 |
| TA − EcN | 6.46 ± 1.84 | 4.51 ± 1.50 | 0.29 ± 0.09 | 1.12 ± 0.40 | 9.71 ± 0.26 | 14.4 ± 0.50 |
| L100 − EcN | 4.48 ± 1.54 | 3.14 ± 0.86 | 0.20 ± 0.08 | 1.14 ± 0.65 | 9.99 ± 0.62 | 14.5 ± 0.90 |
| L100 − TA − EcN | 4.26 ± 1.63 | 2.92 ± 0.91 | 0.25 ± 0.14 | 1.10 ± 0.75 | 9.50 ± 0.30 | 14.1 ± 0.41 |
| L100 − TA − | 5.28 ± 1.95 | 3.23 ± 0.88 | 0.34 ± 0.26 | 0.91 ± 0.55 | 9.87 ± 0.40 | 14.7 ± 0.70 |
| EcN + EDTA | | | | | | |

| | HCT | MCV | MCH | MCHC | PLT |
|---|---|---|---|---|---|
| | (%) | (fl) | (pg) | (g/dL) | (10⁹/L) |
| Reference range | 42.1-68.3 | 50.7-64.4 | 13.2-17.6 | 23.3-32.7 | 420-1698 |
| Healthy control | 53.0 ± 1.77 | 54.8 ± 2.59 | 15.0 ± 0.12 | 28.1 ± 0.99 | 501 ± 21.7 |
| DSS⁺ | 54.7 ± 2.01 | 54.4 ± 0.55 | 14.7 ± 0.21 | 26.9 ± 0.53 | 648 ± 107 |
| EcN | 55.8 ± 2.19 | 56.3 ± 1.75 | 14.9 ± 0.34 | 26.5 ± 0.53 | 609 ± 58.9 |
| TA − EcN | 52.4 ± 2.19 | 54.0 ± 1.75 | 14.8 ± 0.34 | 27.5 ± 1.34 | 626 ± 58.9 |
| L100 − EcN | 53.7 ± 4.34 | 53.7 ± 1.51 | 14.6 ± 0.45 | 27.1 ± 0.76 | 542 ± 56.0 |
| L100 − TA − EcN | 53.6 ± 1.45 | 56.2 ± 0.41 | 14.9 ± 0.15 | 26.4 ± 0.22 | 585 ± 57.8 |
| L100 − TA − | 55.6 ± 2.70 | 56.3 ± 1.51 | 14.9 ± 0.28 | 26.4 ± 0.81 | 550 ± 51.1 |
| EcN + EDTA | | | | | |

WBC, white blood cell; LYM, lymphocyte; MON, monocyte; NEU, neutrocyte; RBC, red blood cell; HGB, hemoglobin; HCT, hematocrit; MCV, mean corpuscular volume; MCH, mean corpuscular hemoglobin; MCHC, mean corpuscular hemoglobin concentration; PLT, platelet. Data are presented as mean±SD (n=5). Reference ranges of hematology data of healthy male BALB/c mice were obtained from Charles River Laboratories: (www.criver.com/).

Histopathology studies. The histopathology analysis for evaluating the colon's damage levels was performed according to standard procedures for paraffin embedding and hematoxylin and eosin (H&E) staining. Briefly, the colonic tissues were fixed in 4% paraformaldehyde solution, embedded in paraffin, sectioned (4 μm), and stained with H&E. The resulting slides were scanned by a Nikon intensilight fluorescence microscope. Each section was scored blindly by a trained pathologist for histological evidence of colon damage by DSS with a scoring system as described in Table 2. See also V. Rees, Chronic experimental colitis induced by dextran sulphate sodium (DSS) is characterized by Th1 and Th2 cytokines. Clinical & Experimental Immunology 114, 385-391 (1998) and K. L. Williams et al., Enhanced survival and mucosal repair after dextran sodium sulfate-induced colitis in transgenic mice that overexpress growth hormone. Gastroenterology 120, 925-937 (2001). The scoring system presented in supporting information included severity of inflammation, depth of injury, crypt damage levels, and the percentage area involved.

TABLE 2
Histological scoring guideline for DSS-induced colitis.

| Inflammation | | Depth of injury | | Crypt damage | | Percentage involved | |
|---|---|---|---|---|---|---|---|
| 0 | None | 0 | None | 0 | None | ×1 | 0~25% |
| 1 | Slight | 1 | Mucosa | 1 | One-third damaged | ×2 | 26%~50% |
| 2 | Moderate | 2 | Mucosa and | 2 | Two-third damaged | ×3 | 51%~75% |
| | | | submucosa | | | | |
| 3 | Severe | 3 | Transmural | 3 | Only surface | ×4 | 76%~100% |
| | | | | | epithelium intact | | |
| | | | | 4 | Entire crypt and | | |
| | | | | | epithelium lost | | |

Statistics. Statistical analysis was evaluated using GraphPad Prism 8. The statistical significance was determined using Student's t-test and one-way ANOVA analysis followed by Tukey's or Fisher's LSD multiple comparison. The differences between experimental groups and control groups were considered statistically significant at P<0.05. (ns) P>0.05, *P<0.05, **P<0.01, ***P<0.001.

### REFERENCES CITED

1. M. E. Sanders, D. J. Merenstein, G. Reid, G. R. Gibson, R. A. Rastall, Probiotics and prebiotics in intestinal health and disease: from biology to the clinic. Nature reviews Gastroenterology & hepatology 16, 605-616 (2019).
2. D.-W. Zheng et al., An orally delivered microbial cocktail for the removal of nitrogenous metabolic waste in animal models of kidney failure. Nature Biomedical Engineering 4, 853-862 (2020).
3. S. Chowdhury et al., Programmable bacteria induce durable tumor regression and systemic antitumor immunity. Nature medicine 25, 1057-1063 (2019).
4. L. Zhang et al., Morphine tolerance is attenuated in germfree mice and reversed by probiotics, implicating the role of gut microbiome. Proceedings of the National Academy of Sciences 116, 13523-13532 (2019).
5. L. V. Blanton, M. J. Barratt, M. R. Charbonneau, T. Ahmed, J. I. Gordon, Childhood undernutrition, the gut microbiota, and microbiota-directed therapeutics. Science 352, 1533-1533 (2016).
6. A. R. Kirtane et al., Nanotechnology approaches for global infectious diseases. Nature Nanotechnology, 1-16 (2021).
7. W. Shin, H. J. Kim, Intestinal barrier dysfunction orchestrates the onset of inflammatory host-microbiome cross-talk in a human gut inflammation-on-a-chip. Proceedings of the National Academy of Sciences 115, E10539-E10547 (2018).
8. D.-W. Zheng et al., Phage-guided modulation of the gut microbiota of mouse models of colorectal cancer augments their responses to chemotherapy. Nature biomedical engineering 3, 717-728 (2019).
9. Q. Zhuang et al., Bacteria-derived membrane vesicles to advance targeted photothermal tumor ablation. Biomaterials 268, 120550 (2021).
10. A. M. Valdes, J. Walter, E. Segal, T. D. Spector, Role of the gut microbiota in nutrition and health. Bmj 361 (2018).
11. J. Li et al., Gastrointestinal synthetic epithelial linings. Science translational medicine 12 (2020).
12. C. Liu et al., Pnictogens in medicinal chemistry: evolution from erstwhile drugs to emerging layered photonic nanomedicine. Chemical Society Reviews (2021).
13. P. W. O'Toole, J. R. Marchesi, C. Hill, Next-generation probiotics: the spectrum from probiotics to live biotherapeutics. Nature microbiology 2, 1-6 (2017).
14. T.-C. Tang et al., Hydrogel-based biocontainment of bacteria for continuous sensing and computation. Nature Chemical Biology, 1-8 (2021).
15. C. Pagnini et al., Probiotics promote gut health through stimulation of epithelial innate immunity. Proceedings of the national academy of sciences 107, 454-459 (2010).
16. J. Suez, N. Zmora, E. Segal, E. Elinav, The pros, cons, and many unknowns of probiotics. Nature medicine 25, 716-729 (2019).
17. B. O. Schroeder, F. Bäckhed, Signals from the gut microbiota to distant organs in physiology and disease. Nature medicine 22, 1079 (2016).
18. A. C. Anselmo, K. J. McHugh, J. Webster, R. Langer, A. Jaklenec, Layer—by —layer encapsulation of probiotics for delivery to the microbiome. Advanced materials 28, 9486-9490 (2016).
19. X. Wang et al., Bioinspired oral delivery of gut microbiota by self-coating with biofilms. Science advances 6, eabb1952 (2020).
20. Y. Lu, A. A. Aimetti, R. Langer, Z. Gu, Bioresponsive materials. Nature Reviews Materials 2, 1-17 (2016).
21. K. Qiu, I. Young, B. M. Woodburn, Y. Huang, A. C. Anselmo, Polymeric Films for the Encapsulation, Storage, and Tunable Release of Therapeutic Microbes. Advanced healthcare materials 9, 1901643 (2020).
22. P. Feng, Z. Cao, X. Wang, J. Li, J. Liu, On—Demand Bacterial Reactivation by Restraining within a Triggerable Nanocoating. Advanced Materials 32, 2002406 (2020).
23. A. M. Vargason, A. C. Anselmo, S. Mitragotri, The evolution of commercial drug delivery technologies. Nature Biomedical Engineering, 1-17 (2021).
24. A. M. Vargason, S. Santhosh, A. C. Anselmo, Surface Modifications for Improved Delivery and Function of Therapeutic Bacteria. Small 16, 2001705 (2020).
25. Q. Hu et al., Conjugation of haematopoietic stem cells and platelets decorated with anti-PD-1 antibodies augments anti-leukaemia efficacy. Nature biomedical engineering 2, 831-840 (2018).
26. P. Dong et al., pH-sensitive Eudragit® L 100 nanoparticles promote cutaneous penetration and drug release on the skin. Journal of Controlled Release 295, 214-222 (2019).
27. W. E. Bentley, G. F. Payne, Nature's other self-assemblers. Science 341, 136-137 (2013).
28. H. Ejima et al., One-step assembly of coordination complexes for versatile film and particle engineering. Science 341, 154-157 (2013).
29. T. Pérez-Berezo et al., Identification of an analgesic lipopeptide produced by the probiotic Escherichia coli strain Nissle 1917. Nature communications 8, 1-12 (2017).
30. T. Danino et al., Programmable probiotics for detection of cancer in urine. Science translational medicine 7, 289ra284-289ra284 (2015).
31. A. Abramson et al., A luminal unfolding microneedle injector for oral delivery of macromolecules. Nature medicine 25, 1512-1518 (2019).
32. S. Lin et al., Copper tannic acid coordination nanosheet: a potent nanozyme for scavenging ROS from cigarette smoke. Small 16, 1902123 (2020).
33. M. Shin et al., Targeting protein and peptide therapeutics to the heart via tannic acid modification. Nature biomedical engineering 2, 304-317 (2018).
34. B. Chassaing, J. D. Aitken, M. Malleshappa, M. Vijay—Kumar, Dextran sulfate sodium (DSS)—induced colitis in mice. Current protocols in immunology 104, 15.25. 11-15.25. 14 (2014).
35. A. F. Bento et al., β-Caryophyllene inhibits dextran sulfate sodium-induced colitis in mice through CB2 receptor activation and PPARγ pathway. The American journal of pathology 178, 1153-1166 (2011).
36. P. Praveschotinunt et al., Engineered E. coli Nissle 1917 for the delivery of matrix-tethered therapeutic domains to the gut. Nature communications 10, 1-14 (2019).

1. A method of encapsulating a probiotic, the method comprising:
(a) overlaying a probiotic with a first layer comprising a compound or composition of matter dimensioned and configured to increase adhesion of the probiotic to intestinal mucosa, wherein the first layer comprises a tannin; and
(b) overlaying the probiotic of step (a) with a second layer comprising a compound or composition that resists degradation in the stomach and at least partially degrades in the small intestine.

2. (canceled)

3. The method of claim 1, wherein the tannin comprise tannic acid.

4. The method of claim 1, wherein the second layer comprises a copolymer comprising monomers selected from the group consisting of methyl acrylate, methyl methacrylate, and methacrylic acid.

5-6. (canceled)

7. The method of claim 1, wherein the probiotic comprises E. coli cells.

8. The method of claim 7, wherein the probiotic comprises Escherichia coli Nissle 1917 cells.

9. A composition of matter made by a method as recited in claim 1.

10. A composition of matter comprising:
a probiotic overlayed with a first layer comprising a compound or composition of matter dimensioned and configured to increase adhesion of the probiotic to intestinal mucosa, wherein the first layer comprises a tannin; and
a second layer overlaying the first layer and comprising a polymer or copolymer that resists degradation in the stomach and at least partially degrades in the small intestine.

11. (canceled)

12. The composition of matter of claim 10, wherein the first layer comprises tannic acid.

13. The composition of matter of claim 10, wherein the second layer comprises a copolymer comprising monomers selected from the group consisting of methyl acrylate, methyl methacrylate, and methacrylic acid.

14-15. (canceled)

16. The composition of matter of claim 10, wherein the probiotic comprises E. coli cells.

17. The composition of matter of claim 16, wherein the probiotic comprises Escherichia coli Nissle 1917 cells.

18. A method of delivering a probiotic to the intestine, the method comprising administering by mouth a composition of matter as recited in claim 10.

19. A method of treating irritable bowel syndrome and colitis, the method comprising administering to a mammal, by mouth, a composition of matter as recited in claim 10, wherein the composition of matter comprises a probiotic in an amount effective to inhibit irritable bowel syndrome and colitis.

20. The method of claim 1, wherein the first layer comprises tannin crosslinked with ferric ions.

21. The method of claim 1, wherein the first layer comprises tannic acid crosslinked with ferric ions.

22. The method of claim 1, wherein:
the first layer comprises tannic acid crosslinked with ferric ions; and
the second layer comprises a copolymer comprising monomers selected from the group consisting of methyl acrylate, methyl methacrylate, and methacrylic acid.

23. The composition of matter of claim 10, wherein the first layer comprises tannin crosslinked with ferric ions.

24. The composition of matter of claim 10, wherein the first layer comprises tannic acid crosslinked with ferric ions.

25. The composition of matter of claim 10, wherein:
the first layer comprises tannic acid crosslinked with ferric ions; and
the second layer comprises a copolymer comprising monomers selected from the group consisting of methyl acrylate, methyl methacrylate, and methacrylic acid.

26. The composition of matter of claim 25, wherein the probiotic comprises E. coli cells.
